(19) United States
(12) Patent Application Publication
(10) Pub. No.: US 2020/0373018 A1
(43) Pub. Date: Nov. 26, 2020

(54) METHOD OF DIAGNOSING PATHOGENESIS OF VIRAL INFECTION FOR EPIDEMIC PREVENTION

(71) Applicant: Yeda Research and Development Co. Ltd., Rehovot (IL)

(72) Inventor: Eran SEGAL, Ramat-HaSharon (IL)

(73) Assignee: Yeda Research and Development Co. Ltd., Rehovot (IL)

(21) Appl. No.: 16/895,449

(22) Filed: Jun. 8, 2020

Related U.S. Application Data
(63) Continuation of application No. PCT/IL2020/050561, filed on May 21, 2020.

(30) Foreign Application Priority Data
Apr. 22, 2020 (IL) 274158

(51) Int. Cl. G16H 50/80; G16H 10/20; G16H 50/70; G16H 50/30

(52) U.S. Cl. CPC G16H 50/80; G16H 50/30; G16H 50/70; G16H 10/20

(57) ABSTRACT

There is provided a method of diagnosing pathogenesis of viral infections for epidemic prevention, comprising: receiving a geographic location and responses to a questionnaire provided to undiagnosed persons, in each of iteration: inputting a first subset of answers and the geographical location of one of the undiagnosed persons into a geographic-level ML model component trained on a first training dataset including, for each subject: the first subset of answers, an indication of a certain geographic zone, and a label indicative of whether the respective subject is diagnosed or undiagnosed with the viral disease, inputting a second subset of the answers to a human-level ML model component trained on a second training dataset including, for each subject, the second subset of answers, and the label, and combining the outcome from the ML model components to calculate a combined likelihood of the respective undiagnosed person to be diagnosed with the viral disease.

### RELATED APPLICATIONS

This application is a Continuation of PCT Patent Application No. PCT/IL2020/050561 having International filing date of May 21, 2020, which claims the benefit of priority of Israel Patent Application No. 274158 filed on Apr. 22, 2020. The contents of the above applications are all incorporated by reference as if fully set forth herein in their entirety.

### FIELD AND BACKGROUND OF THE INVENTION

The present invention, in some embodiments thereof, relates to viral infections and, more specifically, but not exclusively, to systems and methods of diagnosing pathogenesis of viral infections for epidemic prevention.

Viral diseases, for example, COVID-19, may spread rapidly within a population. Early identification of people likely infected with viral disease may help prevent and/or control the spread of the viral disease and prevent full blown epidemics.

### SUMMARY OF THE INVENTION

According to a first aspect, a computer implemented method for diagnosing pathogenesis of viral infections for epidemic prevention, comprises: receiving a plurality of responses to a questionnaire provided to a plurality of undiagnosed persons, each of the plurality of responses comprises a plurality of answers and associated with a geographical location within one of a plurality of geographic zones, in each of a plurality of iterations: inputting a first subset of the plurality of answers and the geographical location of one of the plurality of undiagnosed persons into a geographic-level machine learning (ML) model component trained on a first training dataset including, for each of a plurality of subjects: the first subset of the plurality of answers, an indication of a certain geographic zone of the plurality of geographic zones, and a label indicative of whether the respective subject is diagnosed or undiagnosed with the viral disease, inputting a second subset of the plurality of answers to a human-level ML model component trained on a second training dataset including, for each of a plurality of subjects, the second subset of the plurality of answers, and a label indicative of whether the respective subject is diagnosed or undiagnosed with the viral disease, combining the outcome from the ML model components to calculate a combined likelihood of the respective undiagnosed person to be diagnosed with the viral disease, and treating the respective undiagnosed person for the viral disease according to the combined likelihood using a treatment effective for the viral disease.

According to a second aspect, a computer implemented method for training an ML model for classifying people in multiple geographic areas, optionally for diagnosing pathogenesis of viral infections for epidemic prevention, comprises: obtaining, for each respective subject of a plurality of subjects, a plurality of responses to a questionnaire provided to a plurality of undiagnosed persons, each of the plurality of responses comprises a plurality of answers, an indication of a certain geographic zone of a plurality of geographic zones, and a label indicative of whether the respective subject is diagnosed or undiagnosed with the viral disease, creating a geographic-level training dataset including, for each of the plurality of subjects: a first subset of the plurality of answers, the indication of the certain geographic zone of the plurality of geographic zones, and the label indicative of whether the respective subject is diagnosed or undiagnosed with the viral disease, training the geographic-level ML model component using the geographic-level training dataset, creating a human-level training dataset that includes for each of the plurality of subjects, a second subset of the plurality of answers, and the label indicative of whether the respective subject is diagnosed or undiagnosed with the viral disease, training the human-level ML model component using the human-level training dataset, computing a combination ML model component that combines the outcome from the geographic-level and the human-level ML model components to calculate a combined likelihood of a target undiagnosed person to be diagnosed with the viral disease, and providing the ML model that includes the geographic-level ML model component, the human-level ML model component, and the combination ML model component.

According to a third aspect, a system for classifying people in multiple geographic areas, optionally for diagnosing pathogenesis of viral infections for epidemic prevention, comprises: at least one hardware processor executing a code for: receiving a plurality of responses to a questionnaire provided to a plurality of undiagnosed persons, each of the plurality of responses comprises a plurality of answers and associated with a geographical location within one of a plurality of geographic zones, in each of a plurality of iterations: inputting a first subset of the plurality of answers and the geographical location of one of the plurality of undiagnosed persons into a geographic-level machine learning (ML) model component trained on a first training dataset including, for each of a plurality of subjects: the first subset of the plurality of answers, an indication of a certain geographic zone of the plurality of geographic zones, and a label indicative of whether the respective subject is diagnosed or undiagnosed with the viral disease, inputting a second subset of the plurality of answers to a human-level ML model component trained on a second training dataset including, for each of a plurality of subjects, the second subset of the plurality of answers, and a label indicative of whether the respective subject is diagnosed or undiagnosed with the viral disease, combining the outcome from the ML model components to calculate a combined likelihood of the respective undiagnosed person to be diagnosed with the viral disease, and treating the respective undiagnosed person for the viral disease according to the combined likelihood using a treatment effective for the viral disease.

According to a fourth aspect, a method for diagnosing pathogenesis of viral infections for epidemic prevention and/or classifying people in multiple geographic areas, comprises: receiving a plurality of responses to a questionnaire provided to a plurality of undiagnosed persons, each of the plurality of responses comprises a plurality of answers and associated with a geographical location within one of a plurality of geographic zones, in each of a plurality of iterations: analyzing a first subset of the plurality of answers and the geographical location of one of the plurality of undiagnosed persons, analyzing a second subset of the plurality of answers, combining the outcomes from the analysis to calculate a combined likelihood of the respective undiagnosed person to be diagnosed with the viral disease, and treating the respective undiagnosed person for the viral disease according to the combined likelihood using a treatment effective for the viral disease.

In a further implementation form of the first, second, third, and fourth aspects, the viral disease comprises COVID-19.

In a further implementation form of the first, second, third, and fourth aspects, for at least some of the plurality of undiagnosed persons all of the answers are indicative of lack of any symptoms correlated with the viral disease indicating that each one of the at least some of the plurality of undiagnosed persons are asymptomatic.

In a further implementation form of the first, second, third, and fourth aspects, a test for diagnosing the viral disease comprises PCR.

In a further implementation form of the first, second, third, and fourth aspects, the subject is treated for the viral disease with an effective treatment when the combined likelihood is above a threshold.

In a further implementation form of the first, second, third, and fourth aspects, the viral disease comprises COVID-19 and the effective treatment is selected from the group consisting of: mechanical ventilation, supplemental oxygen, respiratory support, antipyretics, anti-virals, Remdesivir, Oseltamivir, steroids, plasma including antibodies to COVID-19 of subjects that recovered from COVID-19, chloroquine, hydroxychloroquine, and a vaccine against COVID-19.

In a further implementation form of the first, second, third, and fourth aspects, further comprising computing a symptom score by aggregating answers indicative of symptoms, and wherein inputting the first and second subset comprises at least one of: inputting the symptom score, and inputting the symptom score in addition to inputting the first subset and the second subset of answers.

In a further implementation form of the first, second, third, and fourth aspects, the symptom score is computed as a number of positive answers indicative of presence of symptoms, divided by a total number of questions indicative of possible symptoms.

In a further implementation form of the first, second, third, and fourth aspects, the questionnaire includes questions denoting presence of symptoms correlated with population-level likelihood of being infected with the viral disease.

In a further implementation form of the first, second, third, and fourth aspects, presence of the symptoms represented by the plurality of answers to the questions are selected from the group consisting of: no symptoms and feeling good, body temperature, body temperature greater than a threshold, nausea and vomiting, myalgia, rhinorrhea or nasal congestion, fatigue, shortness of breath, cough, sore throat and loss of taste or smell, dry cough, moist cough, chills, confusion, a certain prior medical condition, and diarrhea.

In a further implementation form of the first, second, third, and fourth aspects, the questionnaire includes questions denoting presence of symptoms negatively correlated with population-level likelihood of being infected with the viral disease and positively correlated with population-level likelihood of having another medical condition unrelated to the viral disease.

In a further implementation form of the first, second, third, and fourth aspects, a certain answer to a certain question comprises an age of the undiagnosed subject, and further comprising including the age in at least one of the first subset and second subset.

In a further implementation form of the first, second, third, and fourth aspects, a certain answer to a certain question comprises smoking history and/or presence of chronic medical conditions of the undiagnosed subject, and including the smoking history and/or presence of chronic medical conditions into at least one of the first and second subsets.

In a further implementation form of the first, second, third, and fourth aspects, further comprising receiving for at least some of the plurality of undiagnosed persons, the plurality of answers for a plurality of questionnaires obtained at sequential time intervals, and including the plurality of responses to the plurality of questions for the plurality of questionnaires obtained at sequential time intervals in at least one of the first and second subsets.

In a further implementation form of the first, second, third, and fourth aspects, a combination of non-symptom related questions of the questionnaire denote a unique identifier of each respective undiagnosed person of the at least some of the plurality of undiagnosed persons, and further comprising arranging the responses into the sequential time interval for each respective undiagnosed person according to the unique identifier based on a unique combination of answers to the combination of non-symptom related questions.

In a further implementation form of the first, second, third, and fourth aspects, further comprising receiving an indication of dynamic flow of subjects between the certain geographical zone and at least one other geographical zone, and inputting (and/or analyzing) the indication of dynamic flow into the geographic-level ML model component.

In a further implementation form of the first, second, third, and fourth aspects, the indication of dynamic flow is selected from the group consisting of: traffic patterns, public transportation routes, and walking patterns of subjects.

In a further implementation form of the first, second, third, and fourth aspects, further comprising receiving an indication of dynamic flow of the undiagnosed subject between the certain geographical zone and at least one other geographical zone, and inputting (and/or analyzing) the indication of dynamic flow into at least one of: the geographic-level ML model component and the human-level ML model component.

In a further implementation form of the first, second, third, and fourth aspects, further comprising receiving at least one supplementary static and/or dynamic data, and inputting (and/or analyzing) the at least one supplementary static and/or dynamic data into at least one of: the geographic-level ML model component and the human-level ML model component, wherein the at least one supplementary data is selected from the group consisting of: meteorological data within geographical zones, prescriptions of medications correlated with the viral disease within geographical zones, population density of geographical zones, locations of educational institutions within geographical zones, locations of religious houses of worship within geographical zones, locations of shopping malls within geographical zones, hospitalization of subjects living within geographical zones, subjects assigned to quarantine within geographical zones.

In a further implementation form of the first, second, and third aspects, the outcome from the ML model components is inputted into a combination ML model component that is trained on a third training dataset including, for each of a plurality of subjects, output of the human-level ML component and output of the geographic-level ML component, and a label indicative of whether the respective subject is diagnosed or undiagnosed with the viral disease.

In a further implementation form of the first, second, and third aspects, the first subset of the plurality of answers and the geographical location are inputted into a first processing path comprising the geographic-level ML model component, the second subset of the plurality of answers are inputted into a second processing path comprising the human-level ML model component, and the combination of the outcomes from the ML model components is inputted into a third combined processing path.

In a further implementation form of the first, second, and third aspects, the human level-ML component outputs a human-level likelihood of a certain undiagnosed person likely to be diagnosed with the viral disease.

In a further implementation form of the first, second, and third aspects, the geographic-level ML component outputs at least one of: a geographic-level prediction for number of undiagnosed persons within each respective geographic zone likely to be diagnosed with the viral disease, and a geographic-level prediction for a percent of undiagnosed persons within each respective geographic zone likely to be diagnosed with the viral disease.

In a further implementation form of the first, second, third, and fourth aspects, further comprising: obtaining, for each of the plurality of undiagnosed persons for each of the plurality of geographic zones, a respective combined likelihood, aggregating for each geographic zone the combined likelihoods of undiagnosed persons located within the respective geographic zone, and creating and presenting a coded map indicating, computing a number and/or a percentage of undiagnosed persons likely being diagnosed with the viral disease for each of the plurality of geographic zones.

In a further implementation form of the first, second, third, and fourth aspects, further comprising creating and presenting a coded map indicating the number and/or the percentage of undiagnosed persons likely being diagnosed with the viral disease for each of the plurality of geographic zones.

In a further implementation form of the first, second, third, and fourth aspects, the respective combined likelihood is computed based on the plurality of responses to the questionnaire provided to the plurality of undiagnosed persons on a certain day.

In a further implementation form of the first, second, third, and fourth aspects, further comprising aggregating the combined likelihoods of undiagnosed persons for the plurality of geographic zones for computing a number and/or a percentage of undiagnosed persons likely being diagnosed with the viral disease for a large area consisting of the plurality of geographic zones.

In a further implementation form of the first, and third aspects, a first subset of the plurality of answers and the geographical location of the plurality of undiagnosed persons are inputted into the geographic-level ML model, and a unified geographic-level outcome is obtained from the geographic-level ML model component, and wherein combining comprises combining, for each of the plurality of iterations, the geographic-level outcome from the geographic-level ML model component and each respective outcome from the human-level ML model component, to calculate a respective combined likelihood of the respective undiagnosed person to be diagnosed with the viral disease.

In a further implementation of the fourth aspect, at least one of: a geographic-level prediction for number of undiagnosed persons within each respective geographic zone likely to be diagnosed with the viral disease, and a geographic-level prediction for a percent of undiagnosed persons within each respective geographic zone likely to be diagnosed with the viral disease, is obtained by the analysis of the first subset of the plurality of answers and the geographical location of one of the plurality of diagnosed persons is analyzed.

In a further implementation of the fourth aspect, a human-level likelihood of the one of the plurality of undiagnosed persons likely to be diagnosed with the viral disease is obtained by the analysis of the second subset of the plurality of answers.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart of a method for classifying undiagnosed people in multiple geographic areas during a viral disease outbreak using an ML model, in accordance with some embodiments of the present invention;

FIG. 2 is a block diagram of a system for generating the ML model and/or using the ML model for classifying undiagnosed people in multiple geographic areas, in accordance with some embodiments of the present invention;

FIG. 3 is a flowchart of a method for generating the ML model for classifying undiagnosed people in multiple geographic areas during a viral disease outbreak, in accordance with some embodiments of the present invention;

FIG. 4 is a schematic depicting an overall flow for classification of undiagnosed patients in multiple geographic areas, in accordance with some embodiments of the present invention;

FIG. 5 is an exemplary questionnaire for COVID-19, in accordance with some embodiments of the present invention;

FIG. 6 is a table of characteristics of questionnaire responses received as part of the experiment descried herein, in accordance with some embodiments of the present invention;

FIG. 7 is a graph of prevalence of symptoms for questionnaire responses from neighborhoods in which confirmed cases were presented or no confirmed cases were present, using data collected as part of the experiment descried herein, in accordance with some embodiments of the present invention;

FIG. 8 is a project timeline describing all major events during development as part of the experiment descried herein, in accordance with some embodiments of the present invention;

FIG. 9 is a graph comparing a prediction made based on at least some implementations described herein and close correlation with actual new diagnoses, for multiple cities, in accordance with some embodiments of the present invention;

FIG. 10 is a plot of a prediction made based on at least some implementations described herein versus actual new diagnoses, for multiple cities, in accordance with some embodiments of the present invention;

FIG. 11 is a plot of a trend line over time for all of Israel, for Jerusalem, and for Bene Beraq, in accordance with some embodiments of the present invention;

FIG. 12 is a table depicting correlation of individual symptoms with likelihood of being diagnosed with COVID-19, in accordance with some embodiments of the present invention;

FIG. 13 is a schematic of Average COVID-19-associated symptoms region map based on the experimental results, in accordance with some embodiments of the present invention;

FIG. 14 is a study population flow chart for the second set of experiments, in accordance with some embodiments of the present invention;

FIG. 15 is a table of baseline characteristics of the primary model population for the second set of experiments, in accordance with some embodiments of the present invention;

FIGS. 16A, 16B, 16C, 16D, 16E and 16F are graphs presenting performance of the primary model for the second set of experiments, in accordance with some embodiments of the present invention;

FIG. 17 is a table presenting evaluations for the primary model and the extended features model for the second set of experiments, in accordance with some embodiments of the present invention;

FIGS. 18A-18B are graphs depicting a comparison of the primary model predictions to new COVID-19 cases in Israel over time, for the second set of experiments, in accordance with some embodiments of the present invention;

FIGS. 19A-19B are graphs depicting a feature contribution analysis, for the second set of experiments, in accordance with some embodiments of the present invention;

FIGS. 20A, 20B, 20C, 20D, 20E, 20F, 20G, 20H, 20I and 20J are graphs depicting a feature interpretation analysis, for the second set of experiments, in accordance with some embodiments of the present invention;

FIG. 21 is the online version questions for the COVID-19 survey, for the second set of experiments, in accordance with some embodiments of the present invention;

FIG. 22 is the IVR version questions, for the second set of experiments, in accordance with some embodiments of the present invention;

FIG. 23 is a table presenting COVID-19 diagnosis prevalence and response rate in the IVR cities, for the second set of experiments, in accordance with some embodiments of the present invention;

FIG. 24 is a table presenting baseline characteristics of the extended features model population, for the second set of experiments, in accordance with some embodiments of the present invention; and

FIG. 25 is a chart presenting an odd-ratio (unadjusted) analysis of the primary model population, for the second set of experiments, in accordance with some embodiments of the present invention.

### DESCRIPTION OF SPECIFIC EMBODIMENTS OF THE INVENTION

The present invention, in some embodiments thereof, relates to viral infections and, more specifically, but not exclusively, to systems and methods of diagnosing pathogenesis of viral infections for epidemic prevention.

The present invention, in some embodiments thereof, relates to machine learning (ML) models and, more specifically, but not exclusively, to machine learning models for classifying people in multiple geographic areas during viral disease outbreaks.

As used herein, the term diagnosed and infected may sometimes be interchanged. For example, undiagnosed persons may actually be infected (e.g., being asymptomatic or minimally symptomatic displaying one or few symptoms). The infected but yet undiagnosed persons may be diagnosed as being infected with the infections disease, for example, using a gold standard laboratory test of a physical tissue sample provided by the undiagnosed person. It is noted that false positives and/or false negative test results of the standard laboratory test may be ignored with respect to the embodiments described herein.

An aspect of some embodiments of the present invention relate to systems, methods, an apparatus, and/or code instructions (stored in a memory and executable by one or more hardware processors) for diagnosing pathogenesis of viral infections for epidemic prevention and/or for classifying undiagnosed people in multiple geographic areas (e.g., during an outbreak of a viral disease, for example, COVID-19) using an ML model, for example, as likely to be diagnosed with the viral disease or unlikely to be diagnosed by the viral disease (e.g., where the final diagnosis is made based on laboratory results that analyze tissue samples of the person for presence of the virus). The ML model combines outputs from a geographic-level ML model component that may output geographic-level predictions for a geographic zone as a whole (e.g., number and/or percentage of undiagnosed people likely to be diagnosed, without indicating who those people are) and a human-level ML model component that may output human-level predictions for each respective undiagnosed person (e.g., personal prediction for each person). The ML model components may be independently trained using respective training datasets. The combination is used to calculate a combined likelihood of the respective undiagnosed person to be diagnosed with the viral disease.

Responses to a questionnaire provided to multiple undiagnosed persons are received. Each of the responses includes answers, optionally least some of which are related to the presence of symptoms which are correlated with the viral disease. The responses are associated with a geographical location of the respective undiagnosed person within one of multiple geographic zones. In each of multiple iterations, a first subset of the answers and the geographical location of one of the undiagnosed persons are inputted into a geographic-level ML model component trained on a first training dataset. The first training dataset includes, for each of multiple subjects: the first subset of the answers, an indication of a certain geographic zone of the multiple of geographic zones, and a label indicative of whether the respective subject is diagnosed or undiagnosed with the viral disease. A second subset of the answers is inputted into to a human-level ML model component trained on a second training dataset. The second training dataset includes, for each of the multiple subjects, the second subset of answers, and a label indicative of whether the respective subject is diagnosed or undiagnosed with the viral disease. The outcomes from the ML model components are combined to calculate a combined likelihood of the respective undiagnosed person to be diagnosed with the viral disease. The respective undiagnosed person may be tested for the viral disease and/or treated for the viral disease according to the combined likelihood using a treatment effective for the viral disease.

An aspect of some embodiments of the present invention relate to systems, methods, an apparatus, and/or code instructions (stored in a memory and executable by one or more hardware processors) for training an ML model for classifying undiagnosed people in multiple geographic areas (e.g., during an outbreak of a viral disease, for example, COVID-19) which may be used for diagnosing pathogenesis of viral infections for epidemic prevention, for example, as likely to be diagnosed with the viral disease or unlikely to be diagnosed by the viral disease (e.g., where the final diagnosis is made based on laboratory results that analyze tissue samples of the person for presence of the virus). For each respective subject the following data is received: responses to a questionnaire provided to undiagnosed persons, where each of the responses comprises answers optionally least some of which are related to the presence of symptoms which are correlated with the viral disease, an indication of a certain geographic zone of multiple geographic zones, and a label indicative of whether the respective subject is diagnosed or undiagnosed with the viral disease (i.e., ground truth based on the laboratory test detecting presence of the virus in the tissue sample). A geographic-level training dataset (also referred to herein as the first training dataset) includes, for each of the subjects: a first subset of the answers, the indication of the certain geographic zone, and the label indicative of whether the respective subject is diagnosed or undiagnosed with the viral disease. A geographic-level component of the ML model is trained using the geographic-level training dataset. A human-level training dataset (also referred to herein as the second training dataset) that includes for each of the subjects, a second subset of the answers, and the label indicative of whether the respective subject is diagnosed or undiagnosed with the viral disease. The human-level component of the ML model is trained using the human-level training dataset. Combination code (e.g., a combination ML model component) that combines the outcome from the geographic-level and the human-level ML model components to calculate a combined likelihood of a target undiagnosed person to be diagnosed with the viral disease is computed and/or trained. The ML model that includes the geographic-level ML model component, the human-level ML model component, and the combination code is provided.

An aspect of some embodiments of the present invention relate to systems, methods, an apparatus, and/or code instructions (stored in a memory and executable by one or more hardware processors) for diagnosing pathogenesis of viral infections for epidemic prevention and/or classifying people in multiple geographic areas, using an analysis that is performed by a non-ML component, for example, using other code, and/or manually. Responses to a questionnaire provided to undiagnosed persons, where each of the responses comprises answers and associated with a geographical location within one of multiple geographic zones, is received. In each of multiple iterations: a first subset of the answers and the geographical location of one of the undiagnosed persons is analyzed. Optionally, at least one of: a geographic-level prediction for number of undiagnosed persons within each respective geographic zone likely to be diagnosed with the viral disease, and a geographic-level prediction for a percent of undiagnosed persons within each respective geographic zone likely to be diagnosed with the viral disease, is obtained by the analysis. A second subset of the answers is analyzed. Optionally, a human-level likelihood of the respective one of the undiagnosed persons likely to be diagnosed with the viral disease is obtained by the analysis. The outcomes from the analysis are combined to calculate a combined likelihood of the respective undiagnosed person to be diagnosed with the viral disease. The respective undiagnosed person for the viral disease may be treated according to the combined likelihood using a treatment effective for the viral disease, and/or selected for testing using a standard laboratory test.

Optionally, in some embodiments, the human-level ML component, and/or the geographic-level ML component may be used alone, without necessarily using the other component and/or without necessarily combining outcomes from the other ML component. For example, in some embodiments, the human-level ML component may be used alone, for example, for predicting likelihood of an undiagnosed person to be diagnosed with the viral disease based on a set of questions answered by the undiagnosed person. The set of questions answered by the undiagnosed person may be of symptoms and/or other characteristics of the person (e.g., age, gender, prior medical conditions, smoking habits, and/or geographic location). The set of answers inputted into the ML component used alone may be the full set of responses to the questionnaire, for example, the union of the first subset and the second subset of answers.

At least some implementations of the systems, methods, apparatus, and/or code instructions described herein relate to the technical problem of diagnosing pathogenesis of viral infections for epidemic prevention, by identifying undiagnosed persons that are likely to be diagnosed with a viral disease, for example, with COVID-19. The spread of COVID-19 presents a major challenge to the international community, and policy-makers from different countries have each chosen different strategies, depending on the local spread of the virus, healthcare system resources, economic and political factors, public adherence, and their perception of the situation. One contributing factor to the rise in cases is lack of information about who is infected. Knowing who is likely infected may be used to slow down and/or stop the spread of disease, for example, by isolating those individuals that are likely infected, from other individuals that are uninfected. Lack of being able to identify who is likely infected has led many countries to impose lockdowns (i.e., mandatory isolation) on entire populations, which encounter dramatic side effects, such as economic slowdown, mental health problems from being locked in for prolonged periods of time, and inability to access other medical care for other problems. If individuals that are likely infected are identified, those individuals may be isolated, leaving the non-infected individuals to continue their daily routines. Moreover, identifying who is infected may be used to help protect others who are not yet infected, for example, from being in the same geographical location. Since standard approaches to determining who is infected are based on obtaining a physical tissue sample from the person, and performing a lab test (e.g., PCR) to make a diagnosis of the viral disease, available resources limit the ability to test and sometimes repeatedly test large portions of the population to determine who is infected and who is not infected.

At least some implementations of the systems, methods, apparatus, and/or code instructions described herein provide a technical solution to the above mentioned technical problem, by using answers to questions provided by an undiagnosed person (i.e., for multiple undiagnosed persons), and a geographical location of the undiagnosed person, to predict likelihood of the undiagnosed person being diagnosed with the viral disease, for example, COVID-19. At least some of the answers are related to presence of symptoms. The age of the undiagnosed person may be provided. Other supplemental data may be provided. The responses to the questions and the geographical location are fed into a trained ML model that outputs likelihood of the person being diagnosed with the viral disease. Individuals likely to be diagnosed with the viral disease (e.g., above a threshold) may be selected for testing and/or selected for treatment and/or may be tested and/or may be treated for the viral disease. In this manner, the individuals most likely infected with the viral disease are identified and sent for testing and/or treated, for example, rather than testing large populations, many of which are not infected.

At least some implementations of the systems, methods, apparatus, and/or code instructions described herein provide a technical solution to the above mentioned technical problem, by using two ML model components, trained using different respective training datasets. The training dataset for the geographic-level ML model includes an indication of geographic location. The geographic-level ML model component may analyze data at the geographic zone level, and may generate geographic zone level output, for example, predicted number and/or percent of undiagnosed persons in the geographic zone as a whole being predicted to be diagnosed in the near future (e.g., over the next 0-14 days, which may correspond to the incubation period of the viral disease). Inventors discovered that symptoms and geographic zone are correlated with likelihood of population-level diagnosis values (i.e., infection), for example, as described with reference to the Examples section below. The human-level ML model component may analyze data at the human-level, and may generate human-level output for each respective person. Inventors discovered that some individual symptoms are independently correlated with likelihood of diagnosis (i.e., infection), for example, as described with reference to the Examples section below. The outcomes from the ML model components are combined to calculate a combined likelihood of the respective undiagnosed person to be diagnosed with the viral disease. The respective undiagnosed person may be treated and/or tested for the viral according to the combined likelihood. Inventors discovered that the combination of the outputs of the geographic-level ML model component and the human-level ML model component provide a highly accurate likelihood of a certain target undiagnosed individual to be diagnosed with the viral disease (i.e., infected) when tested with a laboratory test using a physical tissue sample to detect the presence of the virus, for example, as described with reference to the Examples section below.

At least some implementations of the systems, methods, apparatus, and/or code instructions described herein provide technical advantages and/or improvements over other approaches to selecting persons for diagnosis and/or treatment. The persons selected for diagnosis and/or treatment based on the questionnaire and as described herein, are highly likely to be infected, thereby efficiently using available laboratory testing capabilities. Other approaches, for example, testing large populations by obtaining a physical sample from the person and performing PCR on the sample, waste valuable resources since very few such persons may actually be infected. The questionnaire may be easily distributed to large numbers of individuals, for example, accessed by a web browser, and/or obtained as an app installed on a mobile device. Moreover, the questionnaire may be repeatedly administered, for example, daily. Laboratory testing of samples of a large number of individuals on a regular basis, such as daily, cannot be performed with existing available resources.

Other technical advantages and/or improvements over other approaches to diagnosing pathogenesis of viral infections for epidemic prevention and/or by selecting persons for diagnosis and/or treatment include one or more of:
Identifying asymptomatic and/or minimally symptomatic (displaying one or few symptoms, such as two or three) individuals using the questionnaire. For example, younger persons may be infected but asymptomatic or minimally symptomatic. Such persons may be identified as likely to be infected, for example, based on their age and geographic location (e.g., in proximity to many other diagnosed individuals) fed into the ML model. Using the implementations described herein, the asymptomatic and/or minimally symptomatic undiagnosed people may be identified as likely to be diagnosed, and may be sent for laboratory testing and/or treatment and/or laboratory tested and/or treated. In contrast, using standard approaches, such asymptomatic and/or minimally symptomatic undiagnosed people are either ignored (i.e., not treated and/or not tested) or mass testing of entire populations is performed in an effort to find the few individuals that are actually infected.
Differentiate between people infected with COVID-19 and people infected with other viral diseases (e.g., flu) and/or experiencing symptoms due to other medical conditions (e.g., cough due to post nasal drip after a minor cold).
High accuracy in identifying an undiagnosed person as being likely diagnosed with a viral disease (e.g., when tested by a laboratory test using a physical tissue sample to detect the presence of the virus).
Predict future spread per geographic zones before an outbreak occurs, for example, by identifying many undiagnosed individuals in the geographic zone predicted to be diagnosed.

At least some implementations of the systems, methods, apparatus, and/or code instructions described herein address the technical problem of quick diagnosis of viral infections (e.g. COVID-19) and/or without exposure to suspected and/or a mechanism for allocating limited laboratory testing resources. The gold standard for COVID-19 diagnosis is detection of viral RNA in a reverse transcription PCR test. Due to global limitations in testing capacity, effective prioritization of individuals for testing is essential. The rapid and global spread of COVID-19 led the World Health Organization (WHO) to declare it a pandemic on Mar. 11, 2020. One major factor that contributes to the spread of the virus is the apparently large number of undiagnosed infected individuals. This knowledge gap facilitates the silent propagation of the virus, delays the response of public health officials and results in an explosion in the number of cases, for example, as described with reference to Xie J, Tong Z, Guan X, Du B, Qiu H, Slutsky A S. Critical care crisis and some recommendations during the COVID-19 epidemic in China. Intensive Care Med. March 2020. doi:10.1007/s00134-020-05979-7, and Grasselli G, Pesenti A, Cecconi M. Critical Care Utilization for the COVID-19 Outbreak in Lombardy, Italy: Early Experience and Forecast During an Emergency Response. JAMA. March 2020. doi:10.1001/jama.2020.4031. One reason for this knowledge gap is insufficient testing. While the current gold standard for COVID-19 diagnosis is detection of viral RNA in a reverse transcription PCR test, the number of tests is limited by financial and logistic constraints. In a time when almost all countries are faced with the same health challenge, resources are scarce. This creates the need for a prioritization mechanism to allocate tests and resources more efficiently towards individuals who are more likely to test positive, leading to earlier identification of COVID-19 patients and reduced spread of the virus. Despite this need, most countries still employ a simplistic testing strategy based on display of symptoms associated with the disease and either close epidemiological contact with a confirmed COVID-19 case or belonging to a high risk group, for example, as described with reference to Home Page, Ministry of Health. www(dot)health(dot)gov(dot)il/English/Pages/HomePage(dot)aspx(dot) Accessed Apr. 29, 2020. In practice, these strategies lead to a relatively small fraction of positive tests among those tested and thus to inefficient use of the precious testing resources.

At least some implementations of the systems, methods, apparatus, and/or code instructions described herein provide a technical solution to the mentioned technical problem, by providing a tool, which may be used online and/or without the need of exposure to suspected patients, which may be used as part of the prioritization mechanism for selection of patients for diagnosis and/or treatment. Such tool may have worldwide utility in combating COVID-19 by better directing the limited testing resources through prioritization of individuals for testing, thereby increasing the rate at which positive individuals can be identified and isolated. At least some implementations of the systems, methods, apparatus, and/or code instructions described herein provides statistically significant estimates of the probability of an individual to test positive for SARS-CoV-2 infection in a PCR test, based on a national symptom survey that Inventors distributed in Israel. Notably, while most studies describing the clinical characteristics of COVID-19 cases were based on symptoms of hospitalized patients (Zhao et al. 2020), the survey data collected by Inventors allowed Inventors to also study symptoms of milder cases and reveal which symptoms hold the highest predictive power for COVID-19 diagnosis. Using at least some implementations of the systems, methods, apparatus, and/or code instructions described herein, the risk for a positive COVID-19 test can be evaluated in less than a minute and without added costs. At least some implementations of the systems, methods, apparatus, and/or code instructions described herein may be used globally to make more efficient use of available testing capacities, by significantly increasing the fraction of positive tests obtained, and by rapidly identifying individuals that should be isolated until definitive test results are obtained.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

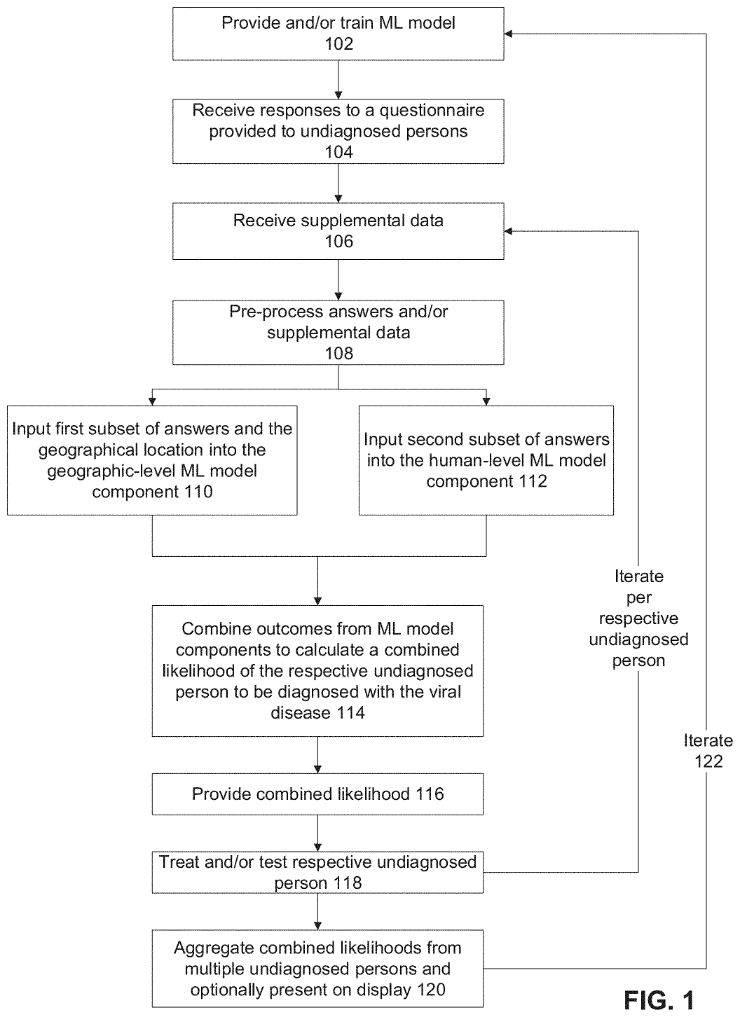

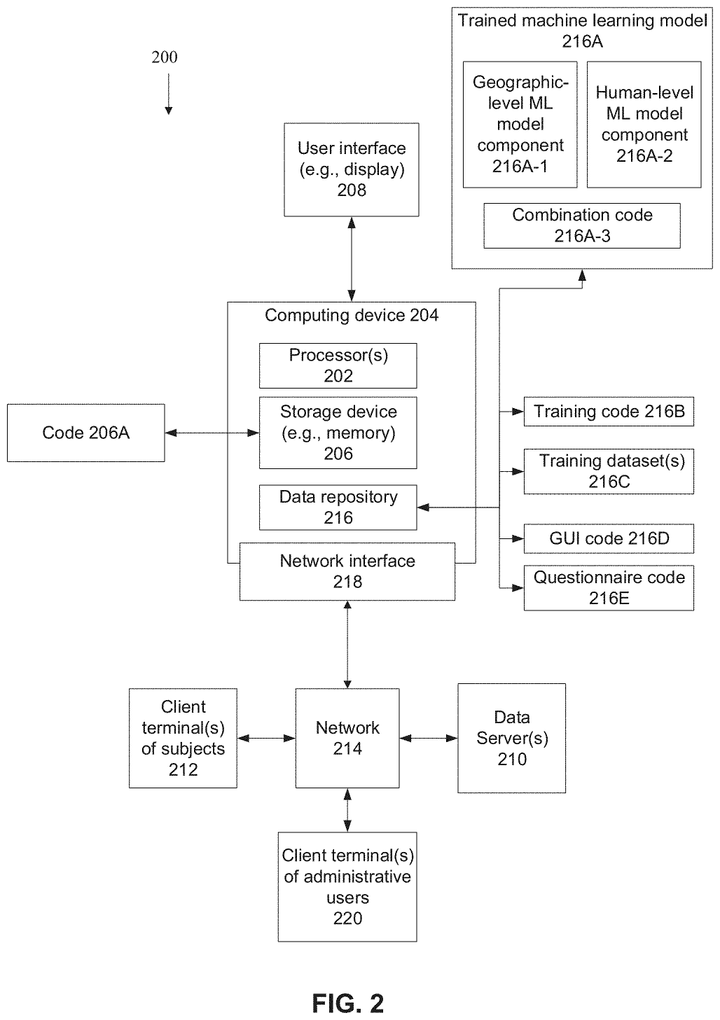

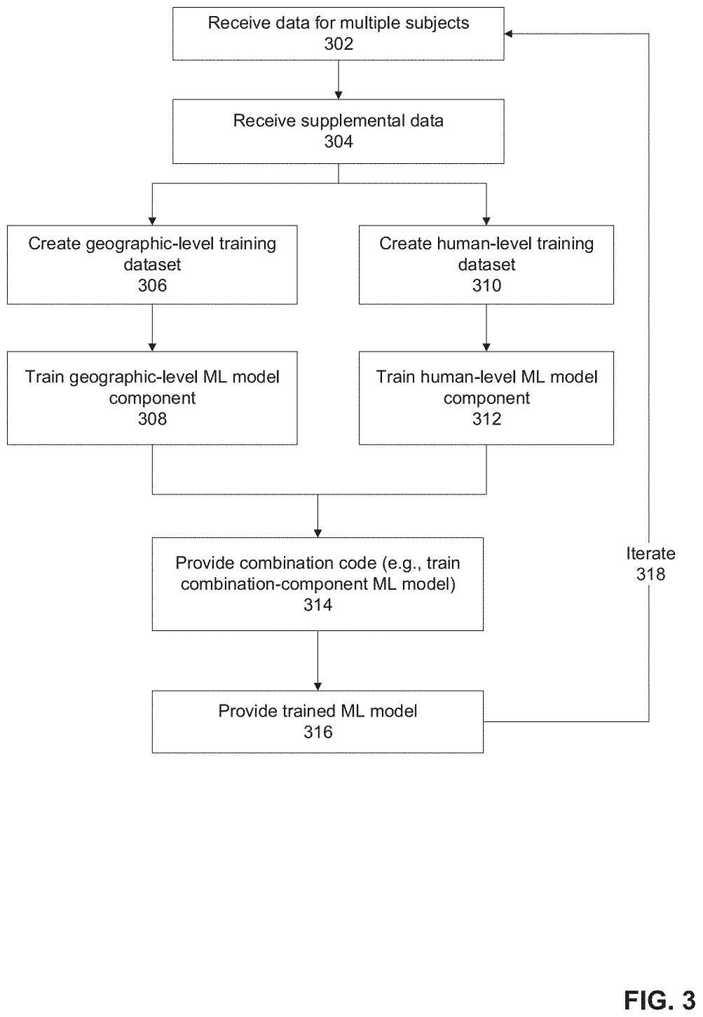

Reference is now made to FIG. 1, which is a flowchart of a method for classifying undiagnosed people in multiple geographic areas during a viral disease outbreak using an ML model, in accordance with some embodiments of the present invention. Reference is also made to FIG. 2, which is a block diagram of a system 200 for generating the ML model and/or using the ML model for classifying undiagnosed people in multiple geographic areas, in accordance with some embodiments of the present invention. Reference is now made to FIG. 3, which is a flowchart of a method for generating the ML model for classifying undiagnosed people in multiple geographic areas during a viral disease outbreak, in accordance with some embodiments of the present invention. System 200 may implement the acts of the method described with reference to FIGS. 1 and/or 3, by processor(s) 202 of a computing device 204 executing code instructions 206A stored in a storage device 206 (also referred to as a memory and/or program store).

Multiple architectures of system 200 based on computing device 204 may be implemented. In an exemplary implementation, computing device 204 storing code 206A may be implemented as one or more servers (e.g., network server, web server, a computing cloud, a virtual server) that provides centralized services (e.g., one or more of the acts described with reference to FIGS. 1 and/or 3) to client terminals of subjects 212 and/or to client terminals of administrative users 220.

Computing device 204 and/or client terminals 212 and/or 220 may be implemented as, for example, a server, a computing cloud, a virtual server, a virtual machine, a mobile device, a desktop computer, a thin client, a Smartphone, a Tablet computer, a laptop computer, a smartphone, a wearable computer, glasses computer, and a watch computer. It is noted that client terminal 212 may be implemented as a standard telephone where responses to questions may be entered by voice (e.g., converted into digital format using voice recognition code) and/or entered by the key pad.

The subjects using client terminals 212 may be people which have been diagnosed with the viral disease, and/or people not diagnosed with the viral disease. The subjects may be symptomatic, asymptomatic, or minimally symptomatic. The subjects answer questions on a questionnaire to provide data which is analyzed by the ML model 216A, as described herein. The client terminals of subjects 212 are used to provide data to computing device 204 by filling out questionnaires, which may be stored as questionnaire code 216E centrally on computing device 204, for example, accessed via a web browser running on client terminals 212. In another example, questionnaire code 216E may be stored locally on each client terminal 212 (e.g., as an app running on a smartphone that is downloaded from code 216E), and/or provided via a software interface that interfaces with code 216E (e.g., application programming interface (API), software development kit (SDK)). Alerts and/or updates may be provided to the respective client terminals 212 by computing device 204, as described herein.

The administrative users using client terminals 220 may be, for example, healthcare providers, policy planners, and/or epidemiologists, which view aggregations of data collected and/or analyzed by computing device 204 using ML model 216A. For example, viewing geographical zone level maps that indicate number and/or percent of undiagnosed persons likely to be diagnosed with the viral infection. Client terminals of administrative users 220 may access computing device 204 to obtain the collected and/or analyzed data, for example, presented within a user interface, optionally a graphical user interface (GUI), such as via a dashboard, as described herein. Client terminals 220 may access GUI code 216D, for viewing the data, as described herein. Client terminals 220 may access GUI code 216D, for example, via a web browser running on client terminals 220. In another example, GUI code 216D may be stored locally on each client terminal 212 (e.g., as an app running on a smartphone that is downloaded from code 216D). In yet another example, the data may be obtained by client terminals 220 from computing device 204, for example, via a software interface (e.g., application programming interface (API), software development kit (SDK)). Alerts and/or updates may be provided to the respective client terminals 220 by computing device 204, as described herein.

In yet another architecture, different client terminals 220 may obtain their own ML models 216A from computing device 204, and/or train their own ML models 216A. For example, adding different layers of supplemental data according to different applications. For example, the ML model used by the army may be trained using different supplemental data than the ML model used by healthcare services.

Supplemental data is provided to computing device 204 from one or more data servers 210. Examples of supplemental data are described with reference to 106 of FIG. 1. The supplemental data may be stored in dataset(s) 216C, and used to train and/or update ML model 216A, as described herein. The supplemental data may be provided to computing device 204 from data server(s) 210, for example, by via API and/or SDK.

Processor(s) 202 of computing device 204 may be implemented, for example, as a central processing unit(s) (CPU), a graphics processing unit(s) (GPU), field programmable gate array(s) (FPGA), digital signal processor(s) (DSP), and application specific integrated circuit(s) (ASIC). Processor(s) 202 may include a single processor, or multiple processors (homogenous or heterogeneous) arranged for parallel processing, as clusters and/or as one or more multi core processing devices.

Data storage device 206 stores code instructions executable by processor(s) 202, for example, a random access memory (RAM), read-only memory (ROM), and/or a storage device, for example, non-volatile memory, magnetic media, semiconductor memory devices, hard drive, removable storage, and optical media (e.g., DVD, CD-ROM). Storage device 206 stores code 206A that implements one or more features and/or acts of the method described with reference to FIGS. 1 and/or 3 when executed by processor(s) 202.

Computing device 204 may include a data repository 216 for storing data, for example one or more of: trained ML model 216A for classification of undiagnosed people, training code 216B for training ML model 216A using training datasets stored in dataset(s) 216C, training dataset(s) 216C that stores data for training ML model 216A including data provided by subjects via questionnaire code 216E and/or supplemental data as described herein, GUI code 216D that presents data, and/or questionnaire code 216E used by subjects to provide data. ML model 216A includes a geographic-level ML model component 216A-1, a human-level ML model component 216A-2, and may include combination code 216A-3 that combines the outcomes of components 216A-1 and 216A-2, optionally implemented as a combination ML model component. Additional details of code 216A-E are described herein. Data repository 216 may be implemented as, for example, a memory, a local hard-drive, virtual storage, a removable storage unit, an optical disk, a storage device, and/or as a remote server and/or computing cloud (e.g., accessed using a network connection).

Computing device 204 may include a network interface 218 for connecting to a network 214, for example, one or more of, a network interface card, a wireless interface to connect to a wireless network, a physical interface for connecting to a cable for network connectivity, a virtual interface implemented in software, network communication software providing higher layers of network connectivity, and/or other implementations.

Network 214 may be implemented as, for example, the internet, a local area network, a virtual private network, a wireless network, a cellular network, a local bus, a point to point link (e.g., wired), and/or combinations of the aforementioned.

Computing device 204 may connect using network 214 (or another communication channel, such as through a direct link (e.g., cable, wireless) and/or indirect link (e.g., via an intermediary computing unit such as a server, and/or via a storage device) with one or more of:
Data server(s) 210, from which the supplementary data is obtained, as described herein.
Client terminal(s) of subjects 212, which provide data via questionnaire code 216E, as described herein.
Client terminals(s) of administrative users 220, which may access computing device 204 to view data, for example, via GUI code 216D, as described herein.

Computing device 204 and/or client terminal(s) 212 and/or 220 include and/or are in communication with one or more physical user interfaces 208 that include a mechanism for a user to enter data (e.g., provide data via the questionnaire) and/or view data (e.g., alerts, geographical maps summarizing data). Exemplary user interfaces 208 include, for example, one or more of, a touchscreen, a display, a keyboard, a mouse, and voice activated software using speakers and microphone.

Referring now back to FIG. 1, at 102, an ML model for classifying people in multiple geographic areas is provided and/or trained. The ML model is designed to be implemented during an outbreak of a certain viral disease. The ML model outputs and/or computes likelihood of a target undiagnosed person to be diagnosed with the viral disease (e.g., diagnosed using a standard laboratory test using a physical tissue sample to detect the presence of the virus). Action may be taken based on the output of the ML model, for example, the target undiagnosed person may be treated for the viral disease, sent for diagnosis using the standard laboratory test, flagged for further observation, and/or instructed to quarantine.

The ML model is trained for a certain viral disease, optionally COVID-19.

It is noted that the ML model may be trained for other infectious diseases that spread from subject to subject, by one subject infecting other subjects, for example, other viruses (e.g., flu), prions, bacteria, parasites, and the like.

The ML model includes the following components: geographic-level component, human-level component, and code that combines outcomes from the ML model components to calculate a combined likelihood of the respective undiagnosed person to be diagnosed with the viral disease, optionally implemented as a combination ML model component.

Optionally, the geographic-level ML model component, the human-level ML model component, and optionally the combination-component are each implemented as respective sub-classifiers.

Each ML model component may be implemented, for example, as one or more of: neural networks of various architectures (e.g., artificial, deep, convolutional, fully connected), Markov chains, support vector machine (SVM), logistic regression, k-nearest neighbor, and decision trees. Sub-classifiers may include portions of the trained classifiers, for example, embeddings may be extracted from hidden layers of neural networks.

Different ML model components may be implemented using different architectures. For example, the geographic-level ML model component and the human-level ML model component are implemented using different architectures.

Optionally, the dataflow through the ML model components is as follows: a first subset of answers and the geographical location (as in 104-108) are inputted into a first processing path including the geographic-level ML model component (as in 110). A second subset of answers (as in 104-108) is inputted into a second processing path including the human-level ML model component (as in 112). The combination of the outputs from the geographic-level and human-level ML model components is inputted into a third combined processing path comprising the combination code (e.g., combination ML model component) (as in 114). Additional details are described herein.

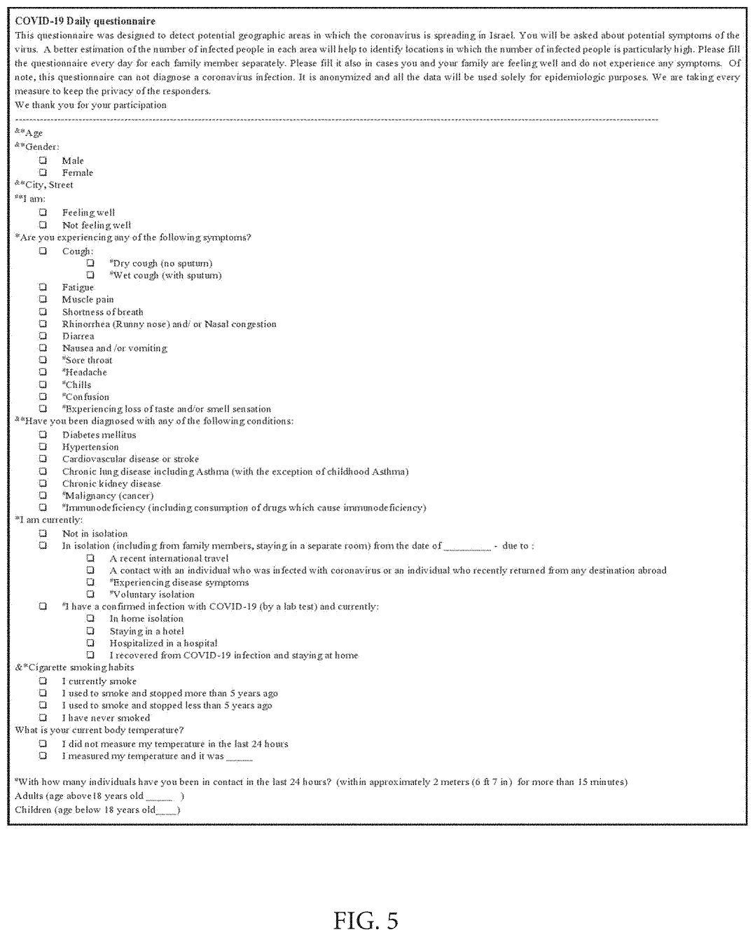

At 104, responses to a questionnaire provided to multiple undiagnosed persons are received. Each of the responses includes answers. For example, the answers are entered by the undiagnosed person via a client terminal and received by the computing device (e.g., server). An exemplary questionnaire is depicted with reference to FIG. 5.

The questionnaire may be accessed, for example, via an app installed on the client terminal (e.g., smartphone), a call to a phone, and/or via a web browser.

The questionnaire may be filled out by the undiagnosed person once. Alternatively, the questionnaire may be filled out by the undiagnosed person at time intervals, for example, daily, once every 2 days, once every 3 days, once a week, and/or triggered by events such as onset of new symptoms and/or disappearance of symptoms. A sequence of questionnaires may be mapped to the same person, for example, by assigning the person a user number (to maintain anonymity), and/or by identifying a subset of questions, where the answers to the subset of questions may be used as a unique identifier of the person. Such questions may include non-symptom related questions. The answers to the non-symptom related questions may remain constant (or relatively constant, such as a time span greater than the incubation period of the virus, such as greater than 1 month, 2 months, or more), enabling the combination of answers to the combination of the non-symptom related questions to be used as the unique identifier. Examples of non-symptom related questions include: age, gender, prior medical conditions, smoking habits and geographical location. Answers to non-symptom related questions are unlikely to change over the time period of an outbreak and/or other monitoring time interval. Responses to questionnaires that received a combination of answers to a combination of the non-symptom related questions may be used as a unique identifier for the undiagnosed person, since the combination of answers may be unlikely to be held by more than one person who answer the questions. Alternatively, the sequence of questionnaires is not mapped to the same person, but treated as independent questionnaires. The user may be prompted to fill out the questionnaire, for example, by a phone call, email, and/or push message.

Each set of responses is associated with a geographical location within one of multiple geographic zones. The geographic location may be provided, for example, entered as an address by the respective undiagnosed person, geo-coordinates obtained from a location sensor of a mobile device used by the respective undiagnosed person (e.g., global positioning sensor (GPS)), from a government database of addressed using another identification of the person, and/or provided by the person when previously answering the questionnaire.

Geographic zones may be defined, for example, according to government divisions, such as cities, neighborhoods, and the like, according to geographical topology, such as based on separations due to rivers, mountains, roads and the like, and/or according to latitude/longitude (or other arbitrary) coordinates, such as dividing a large area into equal sized grid cells. Each geographic zone may include a large number of people (e.g., living therein. Zones may have different numbers of people located therein.

The answers to the questionnaire may be, for example, one or more of: binary (e.g., yes/no), a discrete numerical range (e.g., whole number from 1 to 10), values within a range (e.g., any value from 1-10, including fractions, decimals, and the like), and selection from categories (e.g., happy, sad, flat, joyful).

The questionnaire includes questions denoting presence of symptoms correlated with population-level likelihood of being infected with the viral disease. Exemplary symptoms to which answers are provided include: no symptoms and feeling good, body temperature, body temperature greater than a threshold, nausea and vomiting, myalgia, rhinorrhea or nasal congestion, fatigue, shortness of breath, cough, sore throat and loss of taste or smell, dry cough, moist cough, chills, confusion, a certain prior medical condition (i.e., rather than the presence of any prior medical condition), and diarrhea.

Optionally, some answers are to questions denoting presence of symptoms negatively correlated with population-level likelihood of being infected with the viral disease and positively correlated with population-level likelihood of having another medical condition unrelated to the viral disease. Such answers may be used to help reduce likelihood of the person being diagnosed with the viral disease, for example, the symptom is related to another medical condition.

Optionally, at least some of the undiagnosed persons are asymptomatic, providing answers indicative of lack of any symptoms correlated with the viral disease. Optionally, a mix of sets of answers is received, some from asymptomatic undiagnosed persons, and some from symptomatic undiagnosed persons. As described herein, the ML model may be used to classify asymptomatic undiagnosed persons as likely to be diagnosed with the viral disease.

Optionally, a certain answer to a certain question of the questionnaire includes an age of the undiagnosed person. The age may be included into the first and/or second subsets of answers inputted into the geographic-level ML model component and/or the human-level ML model component.

Optionally, a certain answer to a certain question of the questionnaire includes smoking history and/or presence of chronic medical conditions of the undiagnosed person. The smoking history and/or presence of chronic medical conditions may be included into the first and/or second subsets of answers inputted into the geographic-level ML model component and/or the human-level ML model component.

It is noted that the age and/or smoking history and/or chronic medical conditions may be obtained from another source and inserted into the first and/or subsets, for example, obtained from a storage (e.g., previously provided by the same user) and/or from a database (e.g., health records, government records).

Optionally, for at least some of the undiagnosed persons, a sequence of answers to the same questionnaire (or updated versions of the questionnaire) are obtained at sequential time intervals, for example, daily, once every 2 days, once every 3 days and/or trigged by events such as change in symptoms. The sequential set of answers may be included in the first and/or second subsets which are inputted into the geographic-level ML model component and/or the human-level ML model component.

Optionally, features described with reference to 106-118 may be performed in multiple iterations, optionally for each respective undiagnosed person providing answers to the questionnaire.

Alternatively, features described with reference to 106, 108 and 112-118 may be performed in multiple iterations, optionally for each undiagnosed person providing answers to the questionnaire, and features described with reference to 110 may be performed in a single (or more) iterations for multiple undiagnosed person providing answers to the questionnaire, for example, for the multiple undiagnosed person providing answers to the questionnaire within a time interval, for example, over the course of 24 hours, or 48 hours, or 12 hours, or other intervals.

At 106, supplemental data may be received, for example, from external servers, and/or from a local storage device that stores obtained supplemental data. The supplemental data may increase accuracy of the ML model.

The supplemental data may be associated with individual undiagnosed persons that provided answers to the questionnaire, with a population of multiple undiagnosed persons (optionally per geographic zone), and/or associated with one or more geographic zones of the undiagnosed persons providing answers.

The supplemental data may be a snapshot in time, and/or a sequence of snapshots and/or a pattern (e.g., mathematical model), of dynamic data. The supplemental data may be static data that remains substantially unchanged over long periods of time (e.g., days, weeks, years).

Optionally, the supplemental data includes an indication of dynamic flow of subjects between the certain geographical zone one or more other geographical zone, which may be direct neighbors of one another (e.g., neighborhoods of the same city), or separated by space (e.g., cities on the east and west coast). Such dynamic flow represents a geographic-level and/or population level flow of people. Examples of dynamic flow include: traffic patterns and/or public transportation routes (e.g., cars, busses, planes, ferries), and walking patterns of subjects. The indication of dynamic flow may be inputted into the geographic-level ML model component, optionally added to and/or fed in parallel with, the first subset of answers.

Alternatively or additionally, the supplemental data includes an indication of dynamic flow of the respective undiagnosed person between the certain geographical zone and one or more other geographical zones. The indication of dynamic flow may be inputted into the geographic-level ML model component (optionally added to and/or fed in parallel with, the first subset of answers) and/or into the human-level ML model component (optionally added to and/or fed in parallel with, the second subset of answers).

The dynamic flow data may be obtained, for example, from traffic servers, satellite images, GPS data, traffic navigation applications, and the like. The dynamic flow may be stored, for example, as one or more images and/or videos of traffic movement, metadata (e.g., of routes), and/or descriptions of traffic movement (e.g., destination and origin, vectors and/or functions describing the movement).

Alternatively or additionally, the supplemental data includes one or more of: meteorological data within geographical zones (e.g., temperature, humidity, rain, clouds, for example obtained from weather servers and/or sensors), prescriptions of medications correlated with the viral disease within geographical zones (e.g., obtained from healthcare provider severs), population density of geographical zones (e.g., obtained from government data servers), locations of educational institutions within geographical zones (e.g., obtained from government data servers), locations of religious houses of worship within geographical zones (e.g., obtained from government data servers), locations of shopping malls within geographical zones (e.g., obtained from government data servers), hospitalization of subjects living within geographical zones (e.g., obtained from healthcare data servers), subjects assigned to quarantine within geographical zones (e.g., obtained from healthcare data servers).

Each supplemental data elements may be inputted into the geographic-level ML model component (optionally added to and/or fed in parallel with, the first subset of answers) and/or into the human-level ML model component (optionally added to and/or fed in parallel with, the second subset of answers).

At 108, the answers and/or supplemental data may be pre-processed.

Optionally, the answers (and optionally the supplemental data) are divided into a first and second subset. The first and second subset of answers may overlap, or may be mutually exclusive, or may be identical.

The first subset is designated to be inputted into the geographic-level ML model component, and the second subset is designated to be inputted into the human-level ML model component. The answers (and optionally the supplemental data) included in the first subset correspond to the answers used in the first training dataset (also referred to herein as the geographic-level training dataset) used to train the geographic-level ML model component. The answers (and optionally the supplemental data) included in the second subset correspond to the answers used in the second training dataset (also referred to herein as the human-level training dataset) used to train the human-level ML model component.

Optionally, a symptom score is computed by aggregating answers indicative of presence of symptoms. The symptom score may be computed as a number of positive answers indicative of presence of symptoms, divided by a total number of questions indicative of possible symptoms. For example, when the questionnaire includes yes/no questions for 8 symptoms, and the undiagnosed person provides 3 yes and 5 no answers, the symptom score is computed as 3/8. The symptom score may be computed in other ways, for example, a weighted average of answers each assigned respective weights. For example, some symptoms more indicative of the viral disease may be assigned higher weights.

The symptom score may be may be inputted into the geographic-level ML model component (optionally instead or, or added to and/or fed in parallel with, the first subset of answers) and/or into the human-level ML model component (optionally instead of, or added to and/or fed in parallel with, the second subset of answers).

Optionally, other features are extracted from individual and/or combination of answers and/or supplemental data. Features may be computed, for example, by functions applied to the individual and/or combination of answers. The features may be may be inputted into the geographic-level ML model component (optionally instead or, or added to and/or fed in parallel with, the first subset of answers) and/or into the human-level ML model component (optionally instead of, or added to and/or fed in parallel with, the second subset of answers). Features may be discovered using a feature discovery process.

At 110, the first subset of the answers (which may include a first subset of the supplemental data) and the geographical location of one of the undiagnosed persons is inputted into the geographic-level ML model component.

Optionally, the first subset of answers and the geographic location are inputted into the geographic-level ML model component for each respective undiagnosed person. A respective outcome is obtained for each respective undiagnosed person from the geographic-level ML model, which is combined with the respective outcome from the human-level ML model, as described herein, for example, with respect to 114.

Alternatively, multiple first subsets of answers and multiple geographical locations received from each of the multiple undiagnosed persons are inputted into the geographic-level ML model component, for example, in a single iteration. For example, the first subsets of answers and geographical location received from the multiple undiagnosed persons may be aggregated, for example, concatenated into a single large vector. In another example, the multiple subsets of answers and multiple geographical locations are inputted together, such as simultaneously (or sequentially) into the geographic-level ML model component. A unified geographic-level outcome (e.g., single values or single set of values) is obtained from the geographic-level ML model component. The unified geographic-level outcome may represent an overall geographic-level representation (e.g., prediction) optionally per geographic zone based on the input from multiple undiagnosed persons, for example, how many undiagnosed people are predicted to be diagnosed within the respective zone without necessarily indicating who those people are.

The geographic-level ML model component is trained on a first training dataset including, for each of multiple of subjects: the first subset of the answers, optionally the first subset of supplemental data, an indication of a certain geographic zone of the multiple geographic zones, and a label indicative of whether the respective subject is diagnosed or undiagnosed with the viral disease (e.g., where the diagnosis or non-diagnosis is made by a standard laboratory test such as using physical tissue sample from the subject to detect the presence of the virus) for example, obtained from healthcare provider data that tested the respective subject with a standard laboratory test such as using a physical tissue sample provided by the subject. Each subject is assigned to a certain geographic zone. Subjects are assigned to corresponding geographic zones, for example, according to where the subject is currently located, where subject spends a large amount of time (e.g., working) and/or where subject lives (e.g., according to home address).

The geographic-level ML component may output a geographic-level prediction for number and/or percent of undiagnosed persons within each respective geographic zone likely to be diagnosed with the viral disease. The output is for the geographic zone as a whole, without identifying which subjects are the ones likely to be diagnosed.

One or multiple geographic-level ML model components may be trained. In some implementations, multiple geographic-level ML model components may be trained, each for a certain geographic zone. Each geographic-level ML model component outputs data for one respective geographic zone. In other implementations, a single geographic-level ML model component may be trained, with data labelled per geographic zone. The geographic-level ML model component outputs data for multiple geographic zones.

At 112, the second subset of answers (which may include a second subset of the supplemental data) is inputted into to the human-level ML model component.

Optionally, the geographical location is excluded from the input into the human-level ML model component.

The human-level ML model component is trained on a second training dataset including, for each of the subjects, the second subset of answers, optionally the second subset of supplemental data, and a label indicative of whether the respective subject is diagnosed or undiagnosed with the viral disease.

The human-level ML component may output a human-level likelihood of the respective undiagnosed person likely to be diagnosed with the viral disease. The human-level likelihood is generated for each individual undiagnosed person, as a likelihood measure for that respective person.

At 114, the outcomes from the ML model components (i.e., the geographic-level ML component and the human-level ML components) are combined to calculate a combined likelihood of the respective undiagnosed person to be diagnosed with the viral disease. The combined likelihood may be outputted for example, as a probability value in the range of 0-100%, and/or as a binary value (e.g., likely or unlikely, yes or no), and/or classification category.

Optionally, a respective outcome from the geographic-level ML component obtained for each respective undiagnosed person is combined with a respective outcome from the human-level ML component obtained for each respective undiagnosed person.

Alternatively, the unified geographic-level outcome from the geographic-level ML component obtained for multiple undiagnosed persons is combined with the respective outcome from the human-level ML model component obtained for each respective undiagnosed person. The same unified geographic-level outcome is combined in multiple iterations, where each iteration is using a respective outcome from the human-level ML model obtained for each respective undiagnosed person.

Optionally, the outcomes from the ML model components may include the final output of the respective ML models, optionally, a geographic-level prediction for number and/or percent of undiagnosed persons within each respective geographic zone likely to be diagnosed with the viral disease and/or a human-level likelihood of the respective undiagnosed person likely to be diagnosed with the viral disease.

Alternatively or additionally, the outcomes from the ML model components are obtained from other data feeds of the respective ML model components. For example, when the ML model components are implemented as neural networks, the outcomes may be obtained as embeddings extracted from hidden layers of the neural networks, such as weights of neurons at a certain hidden layer. Such embedding may be stored as vectors. Other examples of outcomes obtained from the respective ML models include intermediate features obtained from intermediate processing stages prior to the final output.

The outcomes may be combined, for example, by concatenation (e.g., of the final output and/or embeddings), by a function, and/or by other mathematical relationships (e.g., mapping).

The outcomes may be combined by inputting the outcomes into a combination ML model component, for example, a classifier, neural network, and/or other architecture as described herein. The combination ML model component may be trained on a third training dataset (also referred to herein as a combination training dataset) that includes, for each of the subjects, output of the human-level ML component, output of the geographic-level ML component, and a label indicative of whether the respective subject is diagnosed or undiagnosed with the viral disease. The combination ML model component calculates the combined likelihood of the respective undiagnosed person to be diagnosed with the viral disease.

At 116, the combined likelihood of the respective undiagnosed person to be diagnosed with the viral disease is provided, for example, stored in a memory, forwarded to another device over the network, and/or fed into another process for additional analysis and/or evaluation, as described herein.

At 118, the respective undiagnosed person is tested (i.e., using a standard laboratory test) and/or treated according to the combined likelihood.

The respective undiagnosed person may be tested and/or treated when the combined likelihood is above a threshold. Persons below the threshold may remain untested and/or untreated. Persons below the threshold may be monitored for trends of computed values for the combined likelihood over time. Persons with increasing trends over time may be tested and/or treated.

The respective undiagnosed person may be tested, for example, using polymerase chain reaction (PCR) or other validated test. The tests may be performed on a tissue sample obtained from the person to detect the presence of the virus.

The respective undiagnosed person may be treated using a treatment that is effective for the viral disease. For example, for COVID-19, one or more (e.g., combination) of the following may be effective treatments: mechanical ventilation, supplemental oxygen, respiratory support, antipyretics, anti-virals, Remdesivir, Oseltamivir, steroids, plasma including antibodies to COVID-19 of subjects that recovered from COVID-19, chloroquine, hydroxychloroquine, and a vaccine against COVID-19.

It is noted that the respective undiagnosed person may be initially selected for testing and/or treatment. Selected people are then tested and/or treated.

Alerts may be sent accordingly, for example, to the undiagnosed person selected for testing and/or treatment, and/or to government and/or healthcare organizations.

At 120, the combined likelihood computed for multiple undiagnosed people is aggregated and/or processed and optionally presented.

Optionally, aggregation is per geographic zone, i.e., for each geographic zone the combined likelihoods of undiagnosed persons located within the respective geographic zone are aggregated. For example, the average combined likelihood is computed per respective geographic zone from the combined likelihoods computed for the people in the respective geographic zone. In another example, a distribution graph and/or chart of combined likelihoods is computed for the people in the respective geographic zone, for example, 20% of the people have a combined likelihood of greater than 70% of being diagnosed, and 50% of the people have a combined likelihood of less than 15% of being diagnosed.

Alternatively or additionally, the aggregation is for multiple geographic zones, or one large zones which may include multiple geographic zones therein, for example, a county, a province, a state, or a country. The number and/or percentage of undiagnosed persons likely being diagnosed with the viral disease for the large area may be computed, for example, a prediction of the number and/or percentage of undiagnosed persons likely being diagnosed with the viral disease for the entire country.

Optionally, the prediction for the large area (e.g., state, country) and/or multiple geographic areas, and/or single geographic area is made based on questionnaires answered during a certain time interval, for example, on a certain date, by aggregating data of the questionnaires answered during the certain time interval.

Optionally, the aggregated data is formatted for presentation, for example, within an interactive user interface, such as an interactive GUI, for example, as a map and/or dashboard. Optionally, a coded map is created, for example, color coded, pattern coded, and/or labelled. The coded map indicates, for each of the geographic zones, a number and/or a percentage of undiagnosed persons likely being diagnosed with the viral disease according to the aggregated combined likelihoods. A user may zoom in to smaller geographical areas. The map may present overlays of the supplemental data, and/or an overlay of an aggregation of the symptom scores and/or other aggregations of answers to the questionnaire.

Optionally, the data is analyzed to determine which questions provide answers (e.g., symptoms, and/or age) have highest predictive power for diagnosis of the viral infection (e.g., COVID-19) contribution of individual questions may be analyzed, for example, using SHAP (SHapley Additive exPlanation). SHAP aims to interpret the output of a machine learning model by estimating the Shapley value of each feature, which represents the average change in the output of the model, by conditioning on that feature while introducing other features one at a time, over all possible features ordering. Analyzing feature contributions in each of the ML model components alone and/or in combination, enables comparing the inner workings of the ML model components alone and/or in combination, to identify which questions dominated each prediction. Inventors discovered that loss of taste or smell, and/or cough (in particular dry cough) may have highest predictive power for diagnosis of COVID-19.

At 122, one or more features described with reference to 102-120 may be iterated. For example, on a daily basis (or other time interval) to obtain daily computations of combined likelihoods for undiagnosed persons.

Iterations may be performed to update the ML model using the laboratory testing results of the undiagnosed persons selected for testing according to the computed combined likelihood. The laboratory test results may serve as ground truth for improving the accuracy of the ML model in computing the combined likelihood.

Referring now back to FIG. 3, at 302, for each respective subject of multiple subjects, the following data is received: responses to a questionnaire designed to be provided to undiagnosed persons, where each of the responses include answers, an indication of a certain geographic zone of geographic zones, and a label indicative of whether the respective subject is diagnosed or undiagnosed with the viral disease (i.e., ground truth obtained by a laboratory test).

At 304, supplemental data may be received. The supplemental data may be associated with individual subjects (e.g., dynamic flow of each individual subject), and/or associated with geographic zones (e.g., meteorological data for each zone). Exemplary supplementary data are described with reference to 106 of FIG. 1.

At 306, a geographic-level training dataset (also referred to herein as a first training dataset) is created. The geographic-level training dataset includes for each of multiple of subjects: a first subset of the answers, optionally a first subset of supplemental data, an indication of a certain geographic zone of the multiple geographic zones, and a label (serving as ground truth) indicative of whether the respective subject is diagnosed or undiagnosed with the viral disease.

Members of the first subset of answers and/or supplemental data may be selected, for example, according to answers and/or supplemental data items that individually and/or as a combination correlate with geographical-level infection (e.g., infection of a population rather with infection of individuals), for example, spreading rates of infection within a geographic zone correlated with meteorological data.

Members may be discovered, for example, by a feature discovery process, manual designation, and/or trial and error processes. Other features may be computed from individual and/or combinations of answers and/or supplemental data items may be discovered and/or extracted.

At 308, the geographic-level component of the ML model is trained using the geographic-level training dataset.

At 310, a human-level training dataset is created. The human-level training dataset includes, for each of the subjects, a second subset of answers, optionally a second subset of supplemental data, and a label (serving as ground truth) indicative of whether the respective subject is diagnosed or undiagnosed with the viral disease.

Members of the second subset of answers and/or supplemental data may be selected, for example, according to answers and/or supplemental data items that individually and/or as a combination correlate with human-level infection (e.g., infection of an individual rather than spread of infection within a population), for example, individuals that are older and have a cough are more likely to be infected than younger individuals with a cough.

Members may be discovered, for example, by a feature discovery process, manual designation, and/or trial and error processes. Other features may be computed from individual and/or combinations of answers and/or supplemental data items may be discovered and/or extracted.

At 312, the human-level component of the ML model is trained using the human-level training dataset.

It is noted that features 306 and 308 may be performed independently from features 310 and 312, for example, in parallel, sequentially, and/or otherwise the generation of one respective training dataset and training of the corresponding ML model component may be performed independently of (e.g., without regards to) the generation of the other respective training dataset and training of the other corresponding ML model component.

It is noted that the architecture and/or implementation of the geographic-level and the human-level training dataset may be different, and/or that the architecture and/or implementation of the geographic-level ML model component and/or the human-level ML model component may be different.

At 314, combination code (e.g., a combination-component of the ML model) is generated and/or defined and/or received. The combination code combines the outcome from the geographic-level and the human-level ML model components to calculate a combined likelihood of a target undiagnosed person to be diagnosed with the viral disease,

The combination code may be trained, for example, using a third training dataset. The third training dataset may be created by including, for each of the subjects, the output of the human-level ML component, the output of the geographic-level ML component, and a label (serving as ground truth) indicative of whether the respective subject is diagnosed or undiagnosed with the viral disease.

Alternatively, the combination code may be defined using other processes and/or based on other analysis processes, for example, manually (e.g., by a domain expert), using regression models, mapping datasets, and/or sets of rules.

At 316, the trained ML model is provided. The ML trained model includes the geographic-level ML model component, the human-level ML model component, and the combination code (optionally combination ML model component).

At 318, one or more features described with reference to 302-316 may be iterated, for example, for updating the ML model using new data, for example, with the laboratory tests results for earlier classified undiagnosed persons, where the actual diagnosis or non-diagnosis result obtained by the laboratory test serving as ground truth to improve the accuracy of the ML model for future classifications.

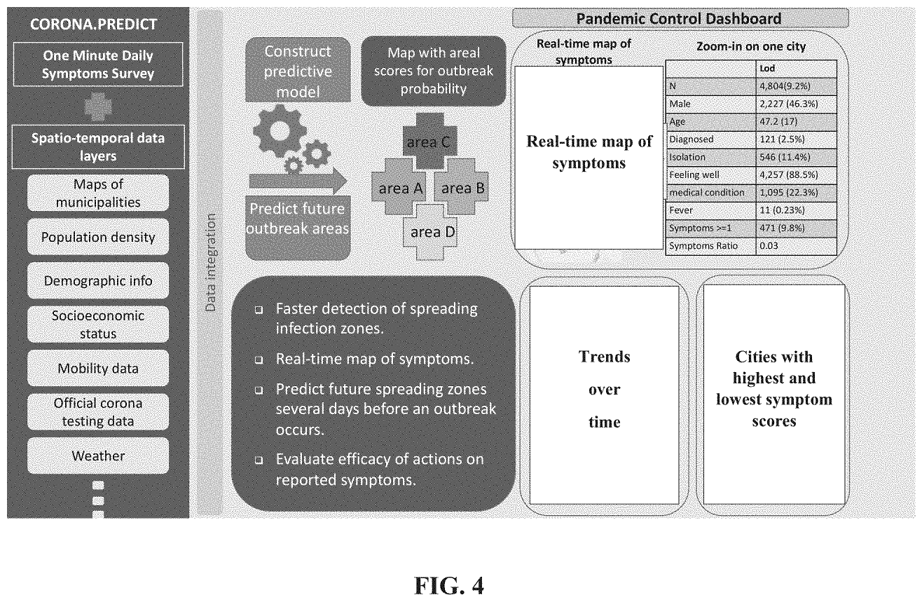

Reference is now made to FIG. 4, which is a schematic depicting an overall flow for classification of undiagnosed patients in multiple geographic areas, in accordance with some embodiments of the present invention.

Various embodiments and aspects of implementations of the systems, methods, apparatus, and/or code instructions as delineated hereinabove and as claimed in the claims section below find experimental support in the following examples.

### EXAMPLES

Reference is now made to the following examples, which together with the above descriptions illustrate some implementations of the systems, methods, apparatus, and/or code instructions described herein in a not necessarily limiting fashion.

Inventors performed several sets of experiments, in accordance with at least some implementations of the systems, methods, apparatus, and/or code instructions described herein.

### First Set of Experiments

In December 2019, a novel coronavirus was isolated, after a cluster of patients in China were diagnosed with pneumonia of unknown cause, for example, as described with reference to Guan, W.-J. et al. N. Engl. J. Med. www(dot)doi(dot)org/10(dot)1056/NEJMoa2002032 (2020). This new isolate was named ‘SARS-CoV-2’ and is the cause of the disease COVID-19. The virus has led to an ongoing outbreak and an unprecedented international health crisis. The number of infected people is rapidly increasing globally, for example, as described with reference to World Health Organization. www(dot)who(dot)int/emergencies/diseases/novel-coronavirus-2019 (accessed 24 Mar. 2020), and most probably is a vast underestimation of the real number of patients worldwide, as infected people are contagious even when minimally symptomatic or asymptomatic, for example, as described with reference to Klompas, M. Ann. Intern. Med. www(dot)doi(dot)org/10(dot)7326/M20-0751 (2020). The spread of the disease has presented an extreme challenge to the international community, and policy-makers from different countries have each chosen different strategies, depending on the local spread of the virus, healthcare-system resources, economic and political factors, public adherence, and their perception of the situation.

As described herein, at least some implementations of the systems, methods, apparatus, and/or code instructions described herein may classify such minimally symptomatic or asymptomatic as being likely to be diagnosed with COVID-19, enabling testing and/or treating those people.

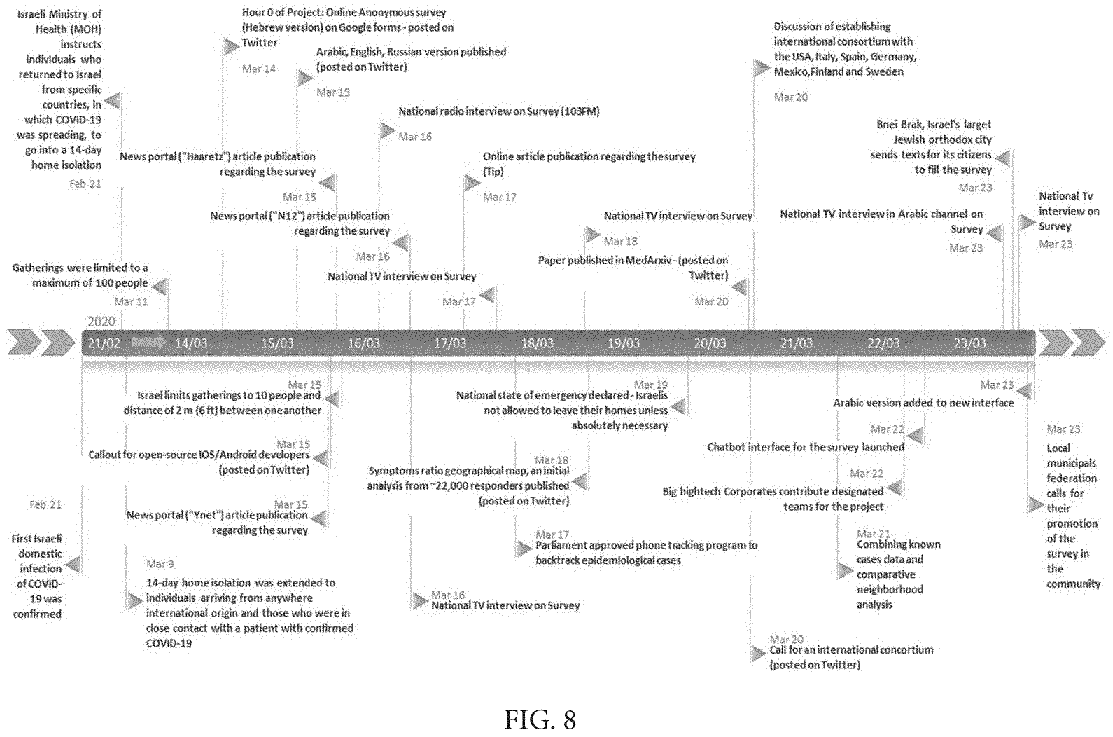

Coronavirus infection spreads in clusters, and early identification of these clusters is critical for slowing down the spread of the virus. As described herein, at least some implementations of the systems, methods, apparatus, and/or code instructions described herein may use daily (or other time intervals) population-wide surveys that assess the development of symptoms caused by the virus as a strategic and valuable tool for identifying such clusters and informing epidemiologists, public-health officials and policymakers. Inventors show preliminary results from an Israeli survey of a cumulative number of over 74,000 responses. At least some implementations of the systems, methods, apparatus, and/or code instructions described herein allow one or more of the following: faster detection of spreading zones and patients; acquisition of a current snapshot of the number of people in each area who have developed symptoms; prediction of future spreading zones several days before an outbreak occurs; and/or evaluation of the effectiveness of the various social-distancing measures taken and their contribution to reducing the number of symptomatic people. This information could provide a valuable tool for decision-makers in those areas in which strengthening of social-distancing measures is needed and those in which such measures can be relieved. Preliminary analysis shows that in geographical zones (i.e., neighborhoods in the experiment) with a confirmed patient history of COVID-19, more people report experiencing COVID-19-associated symptoms, which demonstrates the potential utility of at least some implementations of the systems, methods, apparatus, and/or code instructions described herein for the detection of outbreaks. In Israel, the first infection of COVID-19 was confirmed on 21 Feb. 2020, and in response, the Israeli Ministry of Health (MOH) instructed people who returned to Israel from specific countries in which COVID-19 was spreading to go into a 14-day home isolation. Since then, Israel has gradually imposed several additional measures (See FIG. 8): on 9 March, the 14-day home isolation was extended to people arriving from anywhere of international origin, and those who were in close contact with a patient with confirmed COVID-19 were instructed similarly. Symptomatic people were instructed to stay home for 2 days after symptom resolution, for example, as described with reference to State of Israel Ministry of Health. www(dot)health(dot)gov(dot)il/English/Topics/Diseases/corona/Pages/default(dot)aspx (accessed 24 Mar. 2020). On 11 March, gatherings were limited to a maximum of 100 people; this was further restricted to 10 people on 15 March. On 19 March, a national state of emergency was declared in the country. On 20 March, the first death of an Israeli citizen from COVID-19 occurred.

One of the main challenges of the current pandemic so far has been disease detection and diagnosis. Although the gold standard for the diagnosis of COVID-19 is detection of the virus by a real-time PCR testing, for example, as described with reference to Corman, V. M. et al. Euro Surveill. 25, 2000045 (2020), current resource and policy limitations in many countries restrict the amount of testing that can be performed. The number of tests per day is increasing; however, not enough tests are being performed to provide a nationwide view of the spread of the virus, particularly as the Israeli MOH guidelines are to test only people who were in close contact with a person with confirmed COVID-19.

To obtain a real-time nationwide view of symptoms across the entire population, and since testing the entire population is not feasible, Inventors developed a simple one-minute online questionnaire (also referred to herein as survey) aimed at early and temporal detection of geographic clusters in which the virus is spreading. The survey was posted online (www(dot)coronaisrael(dot)org/) on 14 March, and participants were asked to fill it out on a daily basis and separately for each family member, including members who are unable to fill it out independently (e.g., children and older people). So that potential privacy issues that might occur may be avoided, they survey may filled out anonymously, and access to the data may restricted to only study investigators.

The survey described in the experiment is an exemplary questionnaire used by at least some implementations of the systems, methods, apparatus, and/or code instructions described herein.

The survey contains questions on age, sex, geographic location (city and street), isolation status and smoking habits. Participants also report whether they are experiencing symptoms commonly described in patients with COVID-19 by healthcare professionals, on the basis of the existing literature, for example, as described with reference to Zhao, X. et al. medRxiv www(dot)doi(dot)org/10(dot)1101/2020(dot)03(dot)17(dot)20037572 (2020). Several other symptoms that are less common in patients with COVID-19 but are more common in other infectious diseases are also included to better identify possible patients with COVID-19. The initial symptoms included cough, fatigue, myalgia (muscle pain), shortness of breath, rhinorrhea or nasal congestion, diarrhea and nausea or vomiting. Additional symptoms, including type of cough (with or without sputum), sore throat, headache, chills, confusion and loss of taste and/or smell sensation, were added in a later version. Participants also report about existing chronic health conditions and are asked to report their daily body temperature (see FIG. 5).

Reference is now made to FIG. 5, which is an exemplary questionnaire for COVID-19, in accordance with some embodiments of the present invention. # denotes questions that were added in the new version of the questionnaire and are therefore not analyzed in the experiment described herein. * denotes questions that the participant is required to answer. & denotes questions that should be filled only once.

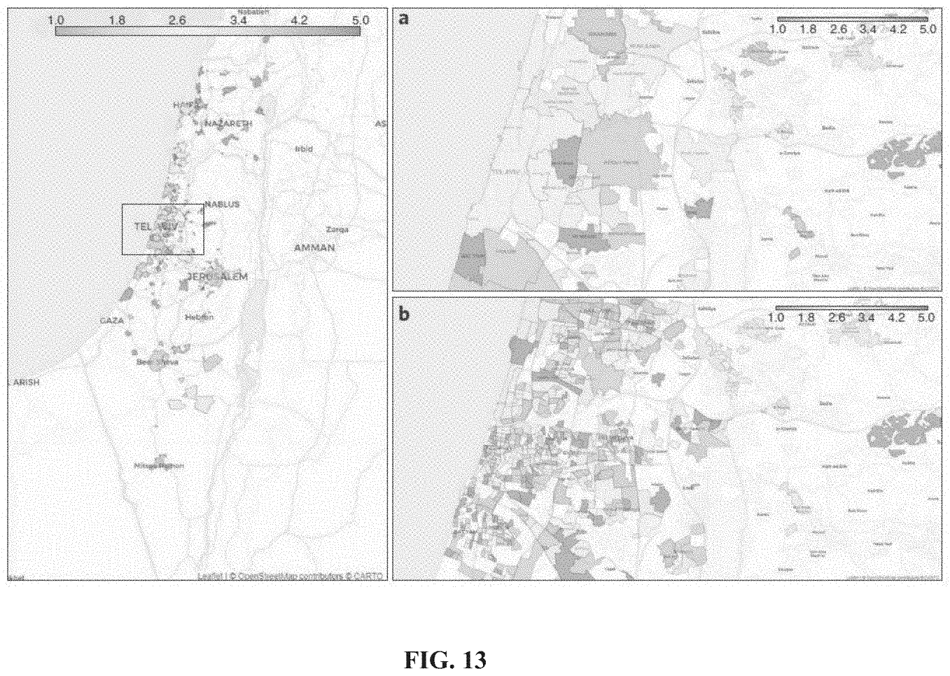

Given that reports on the clinical characteristics of patients with COVID-19 are only starting to emerge, Inventors defined an initial basic measure termed the ‘symptoms ratio’ (also referred to herein as the symptom score, the symptoms ratio is an exemplary implementation of the symptom score) using symptoms that were predefined by the Israeli MOH and are commonly reported by patients with COVID-19, for example, as described with reference to Zhao, X. et al. medRxiv www(dot)doi(dot)org/10(dot)1101/2020(dot)03(dot)17(dot)20037572 (2020). Symptoms assessed were shortness of breath, fatigue, cough, muscle pains and fever (body temperature above 38 degrees Celsius). For participants younger than 18 years of age, nausea and/or vomiting was also included in the ratio calculation. For each participant, the symptoms ratio was calculated as the number of reported symptoms divided by the number of symptoms in our predefined list (number of reported symptoms/6, for participants 18 years of age or less; number of reported symptoms/5, for participants over 18 years of age). The list of symptoms is exemplary, and may be adjusted as more clinical information is accrued. By associating participants with an area corresponding to their address, Inventors created a color map of Israel by the aggregated symptoms ratio in each neighborhood (see FIG. 13).

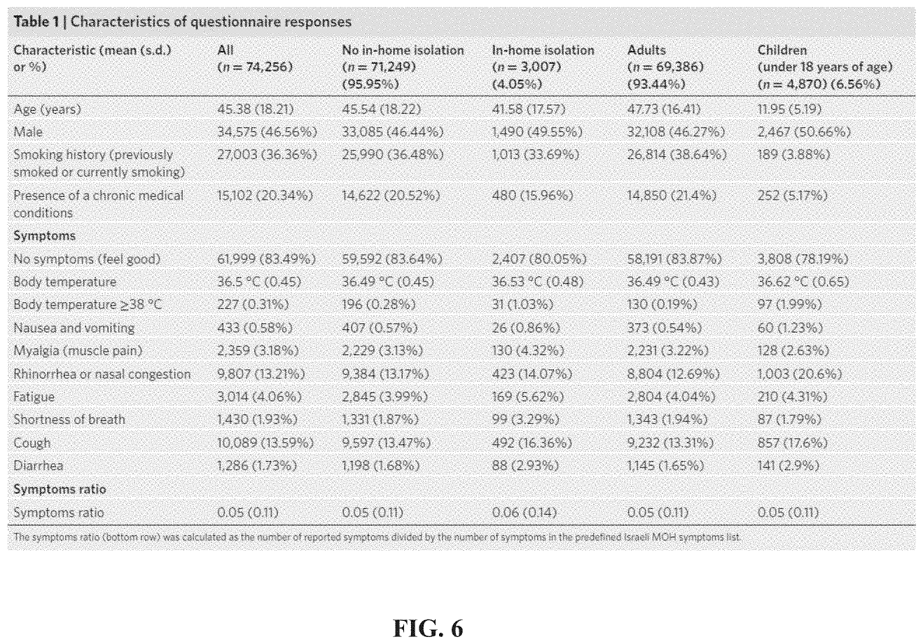

The questionnaire was first distributed online on 14 Mar. 2020, at 14:43 Israel Standard Time (Greenwich Mean Time+2 hours), and was disseminated through social media and traditional press media. As of 23 March, 18:00 Israel Standard Time, a cumulative number of 74,256 responses had been received from 69,386 adults (93.44%) and 4,870 children (6.56%) (see FIG. 6). Of these, 3,007 respondents (4.05%) reported that they were currently in isolation, of which 1,458 (48.49%) were in isolation due to a recent international travel and 1,549 (51.51%) were in isolation due to a contact with a person with COVID-19 or a person who recently returned from abroad. A new version of the questionnaire was established on 21 March, driven by new policies implemented by the Israeli MOH (see FIG. 8) and accumulating data on patients' symptoms, for example, as described with reference to Zhao, X. et al. medRxiv www(dot)doi(dot)org/10(dot)1101/2020(dot)03(dot)17(dot)20037572 (2020). The updated version includes several more questions (see FIG. 5).

Inventors attempted to reach all sectors of the Israeli population in distributing the survey—first, by translating and distributing it in five languages (Hebrew, Arabic, English, Russian and Amharic) that reflect the most common languages spoken in Israel. Second, Inventors devoted efforts to reach underrepresented populations through several channels, including call centers, media appearance and promotion of the survey through Arabic—speaking television stations to gain interest and compliance in all sectors of the population. Inventors analyzed the symptoms ratio of participants by geographical location in Israel (see FIG. 13). This analysis revealed differences in the proportion of reported symptoms in participants from different cities and different neighborhoods that are geographically close to each other, which might suggest the ability to detect changes at high geographical resolution, for example, using the geographic-level ML model component alone, and/or using outcome from the geographic-level ML model component in combination with outcome from the human-level ML component, as described herein.

Inventors analyzed the association between the prevalence of symptoms reported in the survey and the prevalence of the same symptoms in patients with COVID-19, for example, as described with reference to Zhao, X. et al. medRxiv www(dot)doi(dot) org/10(dot)1101/2020(dot)03(dot)17(dot)20037572 (2020). Inventors then integrated data from the Israeli MOH on the locations of known COVID-19 cases and divided the responses into two groups depending on whether they were living in neighborhoods in which confirmed cases were present or not. Notably, in neighborhoods in which people with confirmed COVID-19 were present, Inventors detected a higher prevalence of symptoms that were highly prevalent in patients with confirmed COVID-19 (e.g., cough) and lower rates of symptoms that were less prevalent (e.g., rhinorrhea), which demonstrates the potential of at least some implementations of the systems, methods, apparatus, and/or code instructions described herein for detecting disease clusters at high geographical resolution (see FIG. 8).

In conclusion, the experimental results described herein provide evidence that at least some implementations of the systems, methods, apparatus, and/or code instructions described herein, using a short survey (i.e., questionnaire) developed by Inventors based on symptoms associated with COVID-19 may be used for early detection of clusters of COVID-19 outbreak. It is noted that only 10 days after the survey was first distributed, 74,256 responses had been received. It is noted that Inventors detected a higher percentage of symptoms among people who were in home isolation than among those who were not (0.06 and 0.05, respectively; P=5×10−14 (two-sample t-test)).

Although the spread of COVID-19 is exponential, for example, as described with reference to Li, Y. et al. medRxiv www(dot)doi(dot)org/10(dot)1101/2020(dot)03(dot)01(dot)20029819 (2020), and the number of patients with confirmed COVID-19 in Israel has increased from 193 on 14 March to 1,238 on 23 March, for example, as described with reference to State of Israel Ministry of Health. www(dot)health(dot)gov(dot)il/English/Topics/Diseases/corona/Pages/press-release(dot)aspx (accessed 24 Mar. 2020), the virus has yet to reach the vast majority of Israel's population. Thus, it is possible that the measured symptoms could be reflective of other conditions (such as influenza) that were prevalent in Israel during this period, as many diseases share common symptoms, for example, as described with reference to Zhang, H. et al. Preprints www(dot)doi(dot)org/10(dot)20944/preprints202003(dot)0160(dot)v1 (2020). At least some implementations of the systems, methods, apparatus, and/or code instructions described herein may differentiate between undiagnosed people who are likely infected with COVID-19 (i.e., likely to be diagnosed with COVID-19) and undiagnosed people infected with viruses and/or other diseases other that COVID-19 (i.e., non-COVID-19 infection).

At least some implementations of the systems, methods, apparatus, and/or code instructions described herein provide one or more applications. Although it does not have the ability to directly diagnose individual cases of COVID-19 (since such direct diagnosis may only be made in a laboratory by detecting the actual presence of the virus in a tissue sample), future spreading zones may be predicted a few days before an outbreak occurs, with a high level of accuracy, given a sufficient sample size. Herein is provided a color map of Israel by regions of symptoms ratio (see FIG. 13), which may be adapted to provide predictions of likelihood of infection (i.e., likelihood of being diagnosed) based on the computed likelihoods for multiple undiagnosed people, as described herein.

At least some implementations of the systems, methods, apparatus, and/or code instructions described herein may be leveraged by policymakers to make informed decisions through the utilization of efficient regional prevention strategies rather than a uniform approach. The questionnaire described herein may be used to evaluate the effectiveness of prevention strategies implemented by public-health organizations, such as the various social-distancing measures that are currently being implemented in many countries, including Israel for example, as described with reference to Buckee, C. O. et al. Science www(dot)doi(dot)org/10(dot)1126/science(dot)abb8021 (2020). This may be done by measuring the effect of different strategies on reducing the number of symptomatic people and/or on reducing the combined likelihood of undiagnosed persons to be diagnosed with the viral disease, for example, overall combined likelihood determined for a certain geographical area (e.g., average combined likelihood per respective geographical area). At least some implementations of the systems, methods, apparatus, and/or code instructions described herein may help in elucidating the clinical course of COVID-19 by tracking the dynamics of symptoms and/or combined likelihood in the population and/or in individual people over time.

To improve ease of use by participants and streamline the data-collection process, a designated mobile application may be implemented, as described herein. Privacy issues around location sharing may be resolved in the application, by using the data, for example, only at an aggregated level and may substantially improve accuracy of the ML model, as described herein, and provide valuable insights on population interactions, adherence and disease-spread dynamics, as described herein.

At least some implementations of the systems, methods, apparatus, and/or code instructions described herein may be implemented with various levels of privacy and/or security of personal data. For example, given that participants will be asked for personal medical information, there are concerns about identification and potential misuse of information. As mentioned above, participants fill out the survey anonymously, but location information is also provided (e.g., address details). These data may be secure and made are accessible only by the study investigators. The data may be secured to ensure that the privacy rights of the participants is protected. When the questionnaire is anonymous, the same participant's daily questionnaires cannot necessarily be linked. In other implementations where the same participant's daily questionnaires are linked, individual trends may be identified and/or accuracy of classification may be increased, as described herein. It is noted that sometimes the type of data collected is prone to selection bias. For example, Inventors observed that regions with relatively high response rate are regions associated with higher socioeconomic status. Some bias may decrease as these questionnaires become more widely used and thus better reflect the true population. Bias may be further decreased by adjust for different factors such as age and/or location, and/or by implement national socioeconomic indices.

Reference is now made to FIG. 13, which is a schematic of Average COVID-19-associated symptoms region map based on the experimental results, in accordance with some embodiments of the present invention. It is noted that alternatively or additionally to the symptoms, the map may depict aggregated combined likelihood of undiagnosed people to be diagnosed with COVID-19, as described herein, for example, with reference to 120 of FIG. 1. City municipal regions with at least 30 completed surveys and neighborhoods with at least 10 completed surveys are shown. The color of each region indicates a category defined by the average symptoms ratio, calculated by averaging the reported symptoms rate by responses in that city or neighborhood, but alternatively or additionally may present aggregated combined likelihood values. The values were divided into five categories, and the color of each region indicates its associated category, from green (low symptom rate, which alternatively or additionally may be low aggregated combined likelihood) to red (high symptom rate which alternatively or additionally may be low aggregated combined likelihood) (key). The map on the top right depicts an area of Tel-Aviv and Gush-Dan with city regions. The map on the bottom right depicts area of Tel-Aviv and Gush-Dan with neighborhood regions.

Reference is now made to FIG. 6, which is a table of characteristics of questionnaire responses received as part of the experiment descried herein, in accordance with some embodiments of the present invention.

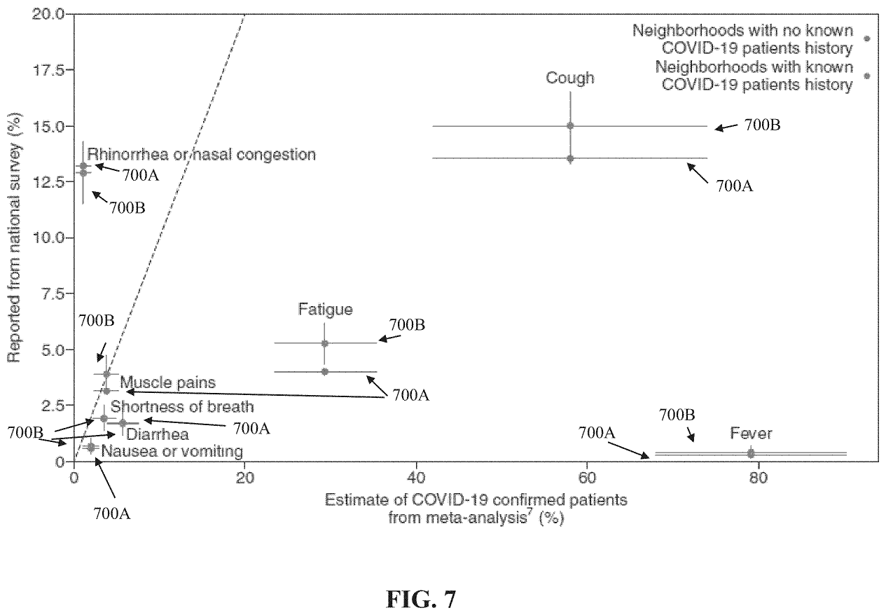

Reference is now made to FIG. 7, which is a graph of prevalence of symptoms for questionnaire responses from neighborhoods in which confirmed cases were presented (red 700B) or no confirmed cases were present (blue 700A), using data collected as part of the experiment descried herein, in accordance with some embodiments of the present invention. Data is presented as estimates and 95% confidence intervals of patients with COVID-19 from a published meta-analysis as described with reference to Zhao, X. et al. medRxiv www(dot)doi(dot)org/10(dot)1101/2020(dot)03(dot)17(dot)20037572 (2020) (x axis) plotted against prevalence from survey data and bootstrap estimates of 95% confidence intervals (y axis); dashed diagonal line, y=x.

Reference is now made to FIG. 8, which is a Project timeline describing all major events during development including national events which affected and drove the process, from time survey online publication (March 14th, 14:44) to March 23rd, March 21, 18:00 IST, as part of the experiment descried herein, in accordance with some embodiments of the present invention.

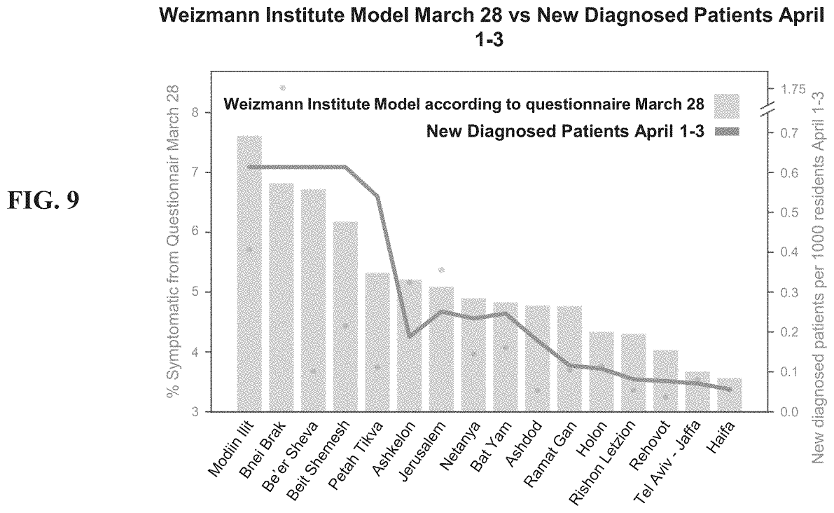

Reference is now made to FIG. 9, which is a graph comparing a prediction made based on at least some implementations described herein (represented as bars) and close correlation with actual new diagnoses (represented as a line), for multiple cities, in accordance with some embodiments of the present invention. The graph provides evidence for the accurate prediction ability of at least some implementations described herein. The data was collected as part of the experiment described herein.

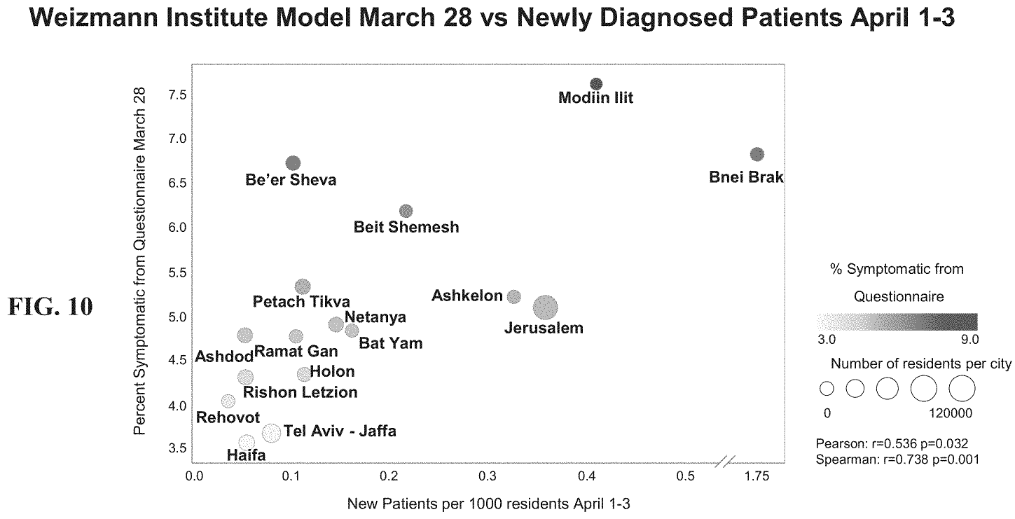

Reference is now made to FIG. 10, which is a plot of a prediction made based on at least some implementations described herein (y-axis) versus actual new diagnoses (x-axis), for multiple cities (respective circles), in accordance with some embodiments of the present invention. The plot provides evidence for the accurate prediction ability of at least some implementations described herein. The data was collected as part of the experiment described herein.

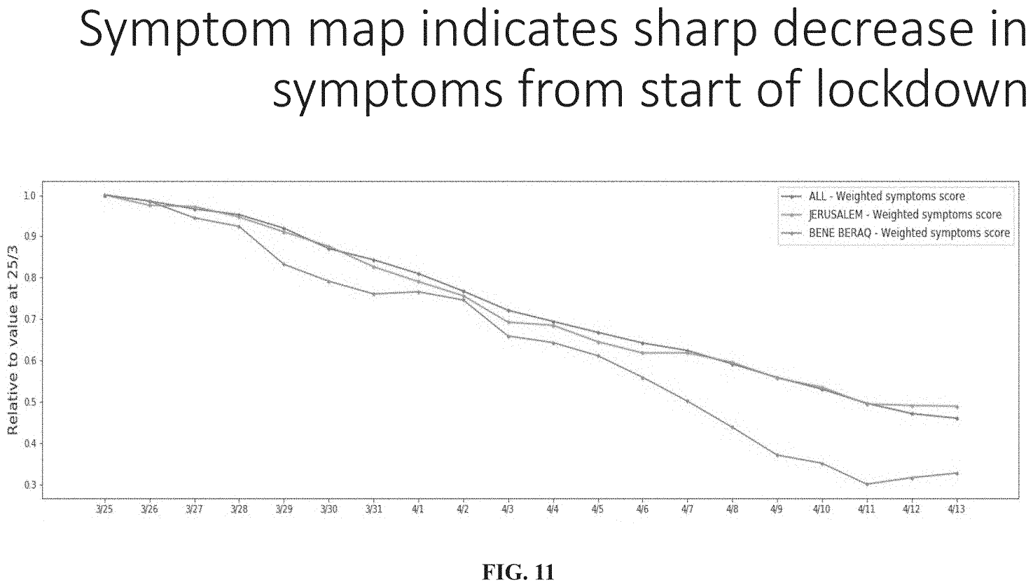

Reference is now made to FIG. 11, which is a plot of a trend line over time for all of Israel, for Jerusalem, and for Bene Beraq, in accordance with some embodiments of the present invention. The trend line is depicted over the course of a mandatory lockdown of the population. The trend line is depicted for a weighted symptom score, but may alternatively or additionally be computed for combined likelihood values aggregated for the respective zones, as described herein. The data was collected as part of the experiment described herein.

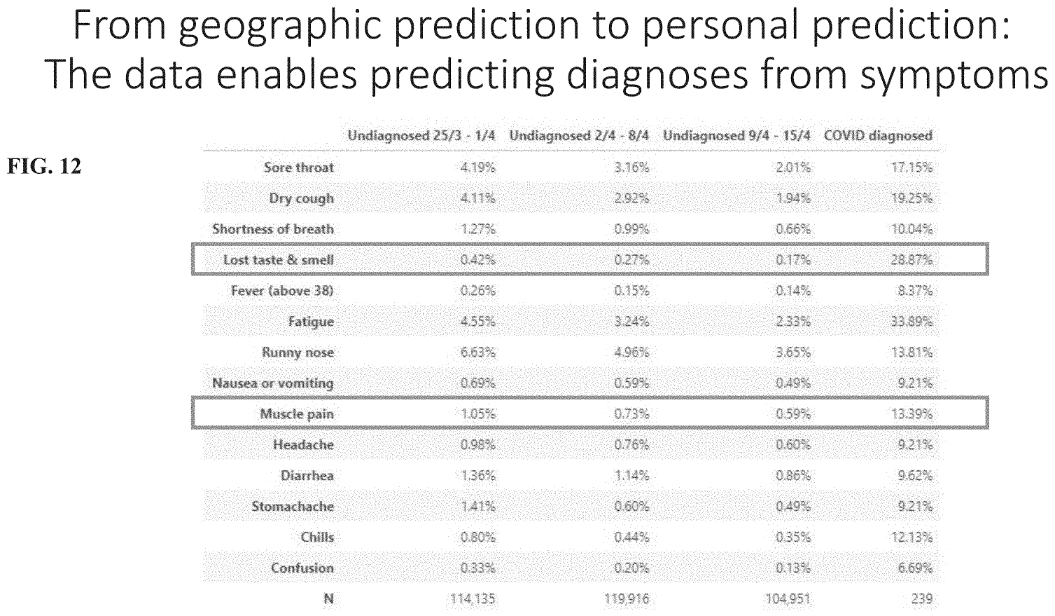

Reference is now made to FIG. 12, which is a table depicting correlation of individual symptoms with likelihood of being diagnosed with COVID-19, in accordance with some embodiments of the present invention. As marked, the symptoms of lost taste and smell, and muscle pain have high correlation with likelihood of an undiagnosed person being diagnosed with COVID-19. The results of the table provide further evidence for the accuracy of the human-level ML component and/or the computed combined likelihood, for individual undiagnosed people. The data was collected as part of the experiment described herein.

### Second Set of Experiments

As described herein, Inventors devised an ML model that estimates the probability of an individual to test positive for COVID-19 based on answers to 9 simple questions regarding age, gender, presence of prior medical conditions, general feeling, and the symptoms fever, cough, shortness of breath, sore throat and loss of taste or smell, all of which have been associated with COVID-19 infection.

The ML model in the experiments may correspond and/or refer to one of more of: the human-level ML model component, the geographic-level ML model component, and/or methods, systems, apparatus, and/or code that combines outcomes from the ML model components, as described herein.

The ML model was devised from a subsample of a national symptom survey that was answered over 2 million times in Israel over the an approximate 2 month period during March-May 2020, and a targeted survey distributed to all residents of several cities in Israel. Overall, 43,752 adults were included, from which 498 self-reported as being COVID-19 positive. The model provides statistically significant predictions on held-out individuals and achieves a positive predictive value (PPV) of 51.8% at a 10% sensitivity. Its predictions on every date also forecasted the number of COVID-19 confirmed cases identified four days later with high accuracy (R=0.9). As a tool based on the ML model may be used online and without the need of exposure to suspected patients, such online tool may have worldwide utility in combating COVID-19 by better directing the limited testing resources through prioritization of individuals for testing, thereby increasing the rate at which positive individuals can be identified and isolated.

### Methods

### Data

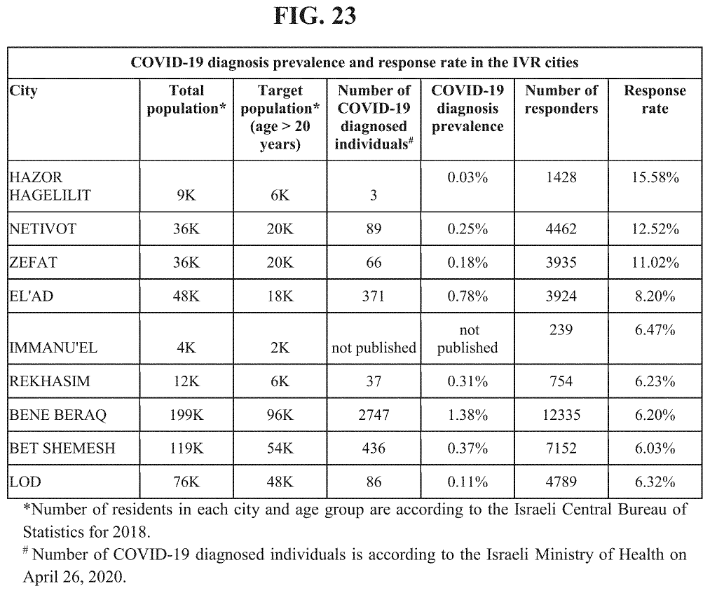

Inventors utilized data that originates from two versions of a one-minute survey that was developed and deployed by Inventor's research group in the early stages of the COVID-19 spread in Israel, for example, as described with reference to Nat Med. April 2020. doi:10.1038/s41591-020-0857-9. The online version of the survey includes questions relating to age, gender, prior medical conditions, smoking habits, self-reported symptoms and geographical location. Questions regarding prior medical conditions and symptoms included in the survey were carefully chosen by medical professionals. Each participant is asked to fill the survey once a day for himself and for family members that are unable to fill it for themselves (e.g., children and the elderly). The survey is anonymous to maintain the privacy of the participants, and has been collected since Mar. 14, 2020. As the number of COVID-19 diagnosed individuals in Israel rose, in some cities more than others, a shortened version of the survey was deployed using an Interactive Voice Response (IVR) platform. This version of the survey included information on respondents' age group, gender, presence of prior medical conditions, general feeling and a partial list of symptoms, including fever, cough, shortness of breath, sore throat and loss of taste or smell. Cities were targeted to participate in the IVR version of the survey according to the number of diagnosed patients and an increased concern for COVID-19 outbreaks (see FIG. 23). Starting Apr. 5, 2020, citizens in the targeted cities were contacted and responses were collected anonymously.

### Unique Identifier

The IVR version of the survey was collected once in each selected city, and thus each responder was questioned once. In the online version of the survey, individuals were encouraged to respond daily, but since responses are anonymous, repeated answers from the same individual cannot be strictly identified. To allow the construction of an integrated data set from both versions of the survey, without repeating answers from the same individuals, Inventors defined a subset of questions which determine a unique identifier for every response recorded in the online survey. These include information on age, gender, prior medical conditions, smoking habits and geographical location—as answers to these questions are unlikely to change over the time period of our study. Responses that received the same unique identifier were treated as if they were answered by the same individual.

### Study Design and Population

Overall, 695,586 and 66,447 responses were collected up until Apr. 26, 2020, from the online and IVR versions of the survey, respectively. Since children express different clinical manifestations of COVID-19 infection, for example, as described with reference to Dong Y, Mo X, Hu Y, et al. Epidemiological characteristics of 2143 pediatric patients with 2019 coronavirus disease in China. Pediatrics. March 2020. doi:10.1542/peds.2020-0702, and de Souza T H, Nadal J A, Nogueira R J N, Pereira R M, Brandao M B. Clinical Manifestations of Children with COVID-19: a Systematic Review. medRxiv. April 2020. doi:10.1101/2020.04.01.20049833, Inventors decided to focus the analysis only on adults (age above 20 years old). To avoid translation discrepancies, Inventors included only responses in Hebrew. Inventors also excluded responses that did not meet quality control criteria, such as a reasonable age (>120 years old) and body temperature (35-43° C.) or responses suspected as spam.

To ensure data reliability, responses were filtered by the following criteria:

Online version:
Age below 0 or above 120 years old
Body temperature below 35 or above 43
The same unique identifier within a period of 1 hour
More than 6 positive symptoms
More than 4 prior medical conditions

IVR Version:
More than 3 missing answers
More than 5 positive symptoms or prior medical conditions

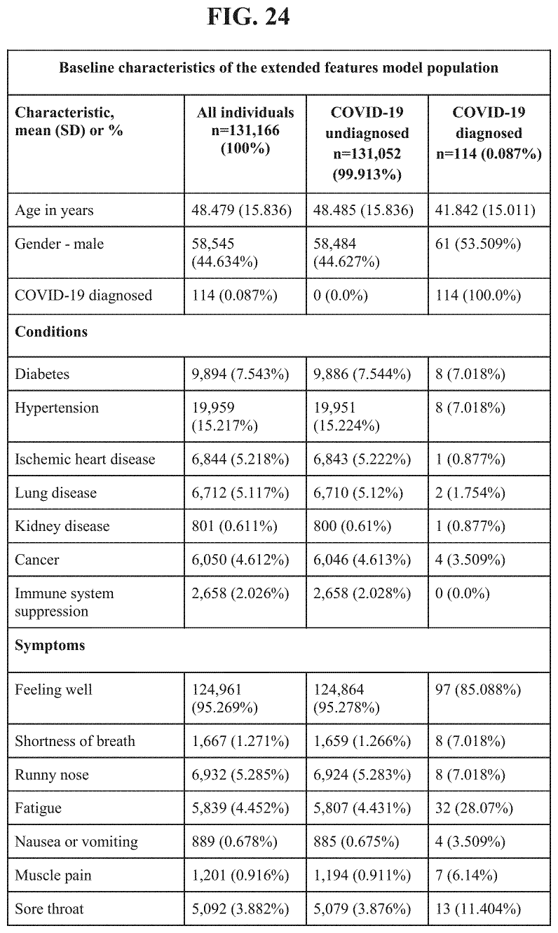

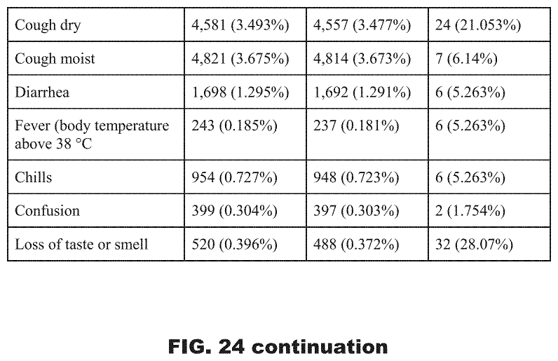

A total of 33,737 responses were eventually included from the IVR version of the survey. Since surveyed cities were at relatively high risk (see FIG. 23), the prevalence of COVID-19 diagnosed responders in the IVR was 1.14%, 6 times higher than the national prevalence of 0.18%, for example, as described with reference to Home Page, Ministry of Health. www(dot)health(dot)gov(dot)il/English/Pages/HomePage(dot)aspx(dot) Accessed Apr. 29, 2020. These cities also had very high response rates, between 6% to 16% of the cities' population (see FIG. 23). From the online version of the survey, Inventors randomly sampled a single response for each individual, and when an individual reported a COVID-19 diagnosis, Inventors randomly sampled one response from those included a positive diagnosis answer. A total of 131,166 responses were identified in the online version, of which 0.09% reported a positive COVID-19 diagnosis, which is closer to the national prevalence of 0.18%, for example, as described with reference to Home Page, Ministry of Health. www(dot)health(dot)gov(dot)il/English/Pages/HomePage(dot)aspx(dot) Accessed Apr. 29, 2020. The characteristics of these unique responders are described in FIG. 24.

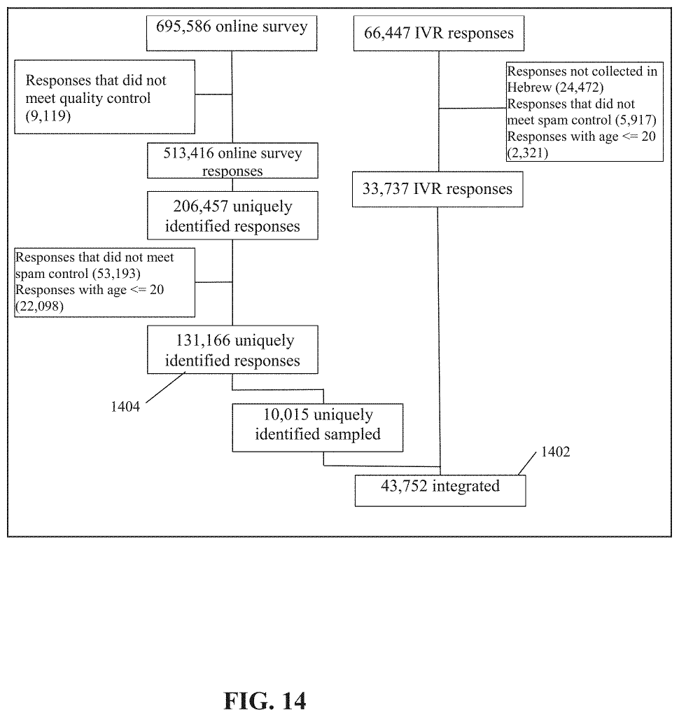

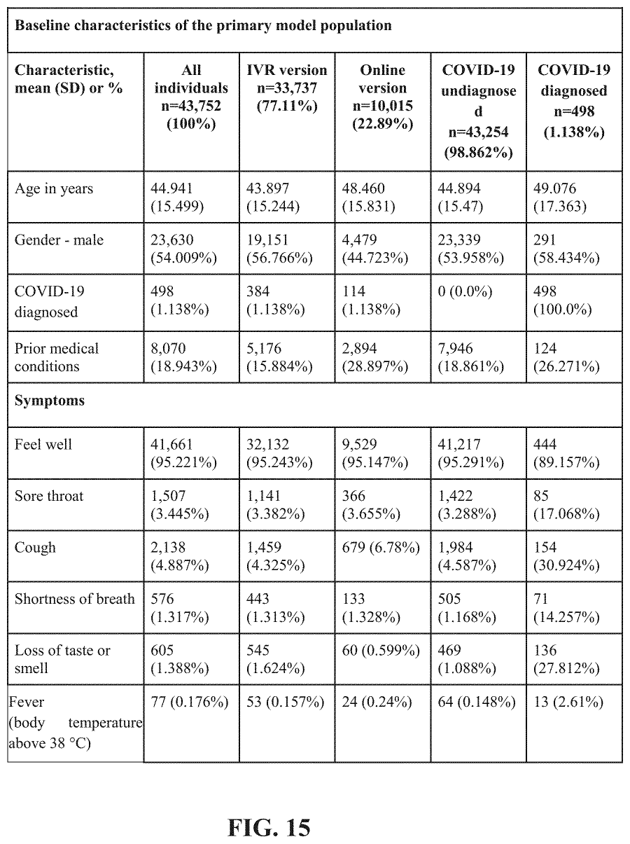

For the integration of the two survey versions, all 33,737 IVR responses were combined together with all 114 uniquely identified responders in the online survey that self-reported COVID-19 diagnosis and a random sample of 9901 undiagnosed responders in the online version, to maintain the same diagnosis prevalence as in the IVR version (see FIG. 14). Overall, 43,752 responses were eventually included in the study, of which 498 self-reported as being COVID-19 diagnosed. The characteristics of these responders are described in FIG. 15.

### Predicting the Outcome of a COVID-19 Test

Inventors defined survey self-reporting of a COVID-19 laboratory confirmed diagnosis as an outcome. Inventors constructed two ML models. The first, which is referred to herein as the primary model, was constructed from the responses from both the IVR and online surveys and included the reduced set of questions that were surveyed in the IVR version. The second ML model, which is referred to herein as the extended features model, was constructed using only responses from the online version, and included additional symptoms and questions that were not part of the IVR survey. Inventors trained both the primary and extended features models using Logistic Regression. In addition, in order to capture nonlinear interactions and interactions amongst features, in both cases Inventors also constructed models using a Gradient Boosting Decision Trees algorithm, for example, as described with reference to Chen T, Guestrin C. XGBoost: A Scalable Tree Boosting System. In: Proceedings of the 22nd ACM SIGKDD International Conference on Knowledge Discovery and Data Mining—KDD '16. New York, N.Y., USA: ACM Press; 2016:785-794. doi:10.1145/2939672.2939785. To test the validity of the first and second models, all model constructions were done using the framework of cross-validation, in which model performance is evaluated on a subset of the data that was not used in model construction.

The logistic Regression models (for the purposes of the Experiment, representing one exemplary, not necessarily limiting, implementation) were constructed using 4 folds cross-validation and features were imputed with the most frequent answer in each feature. Gradient Boosting Decision Trees models (for the purposes of the Experiment, representing one exemplary, not necessarily limiting, implementation) were constructed using a double nested cross-validation, with 4 folds for cross-validation prediction and 2 folds for parameter tuning. Primary model parameters are: colsample_bytree 0.75, learning_rate 0.005, max_depth 4, min_child_weight 7.5, n_estimators 500, subsample 0.8, and extended features model parameters are: colsample_bytree 0.75, learning_rate 0.005, max_depth: 4, min_child_weight 10, n_estimators 1250, subsample 0.75. Since the only question that responders were allowed to skip was body temperature, and the answer to this question is unlikely to be missing at random, meaning that people that did not measure their body temperature are more likely to not have high fever, Inventors imputed this feature with the equivalent answer of fever under 38° C. sklearn version: 0.21.3 and xgboost version: 1.0.2 were used.

### Primary Model

The primary model was constructed using responses to both the IVR and online surveys. Features included in this model were determined by the IVR version, since it included a subset of the online version questions. These consisted of age group, gender, presence of prior medical conditions, general feeling, and the following symptoms: fever, cough, shortness of breath, sore throat and loss of taste or smell.

### Extended Features Model

The extended features model was constructed using only responses from the online version of the survey, as it had 14 additional features that were not available in the IVR version. This extended list added dry cough and moist cough (instead of general cough in the primary model), fatigue, muscle pain, rhinorrhea, diarrhea, nausea or vomiting, chills, confusion and reporting on presence of specific prior medical conditions separately (as opposed to the presence of any prior medical condition in the primary model).

### Baseline Models

To assess the contribution of reported symptoms and prior medical conditions to both the primary model and the extended features model (in both the Logistic Regression and the Gradient Boosting Decision Trees versions), Inventors constructed baseline models using only age group and gender information to predict the outcome.

### Analysis of Model Feature Contributions

To gain insight into the features that contribute most to the predicted probability of being diagnosed with COVID-19 of the models, Inventors analyzed feature contribution in the Gradient Boosting Decision Trees models using SHAP (SHapley Additive exPlanation), for example, as described with reference to Lundberg S M, Erion G, Chen H, et al. From local explanations to global understanding with explainable AI for trees. Nat Mach Intell. January 2020. doi:10.1038/s42256-019-0138-9. SHAP aims to interpret the output of a machine learning model by estimating the Shapley value of each feature, which represents the average change in the output of the model, by conditioning on that feature while introducing other features one at a time, over all possible features ordering. Analyzing feature contributions in each of the models allowed us to compare the inner workings of each model and to identify which features dominated each prediction.

Inventors further analyzed SHAP interaction values, which uses the ‘Shapely interaction index’ to capture local interaction effects between features, for example, as described with reference to Lundberg S M, Erion G, Chen H, et al. From local explanations to global understanding with explainable AI for trees. Nat Mach Intell. January 2020. doi:10.1038/s42256-019-0138-9. Interaction values are calculated for each pair of the model's features, and for each individual prediction of the model, allowing uncovering interaction patterns between pairs of features. Inventors placed particular emphasis on the contribution of the age of participants and the interaction of age with all other symptoms.

Results

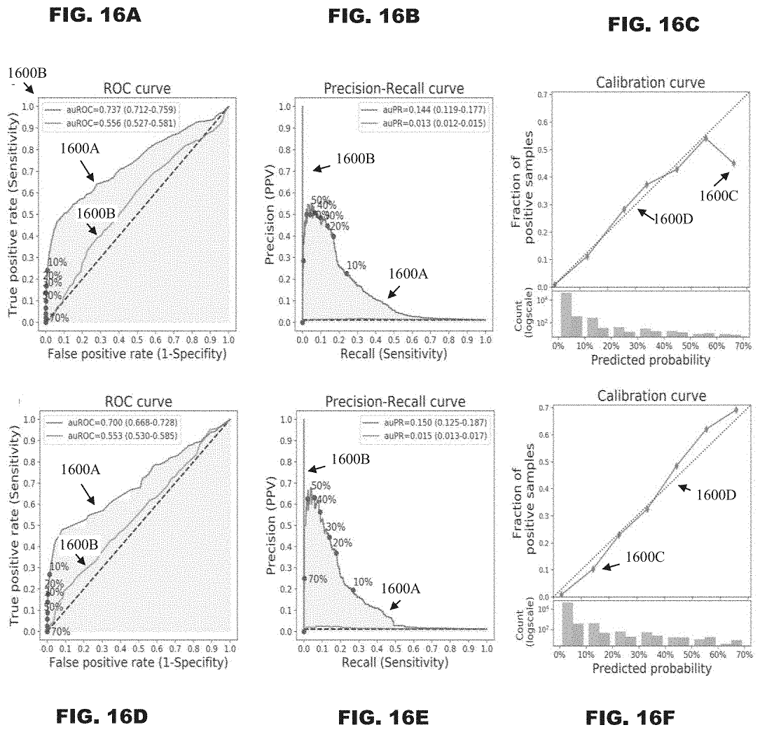

The primary model for prediction of a positive COVID-19 test result, which was constructed using Logistic Regression, achieved an area under the Receiver-Operating-Characteristic (auROC) of 0.737 (CI: 0.712-0.759), and an area under the Precision-Recall (auPR) of 0.144 (CI: 0.119-0.177). This model significantly outperforms the baseline model, which uses only age group and gender (see FIG. 16A-B). Aside from discrimination performance measures, Inventors also tested whether the model was calibrated. In a perfectly calibrated model, the distribution of the predicted probabilities is equal to the distribution of outcomes observed in the training data. Inventors found that our primary model is well calibrated across the relevant prediction range (see FIG. 16C). The model has a positive predictive value (PPV) of 45.2%, at 10% sensitivity and 24.8% at 20% sensitivity.

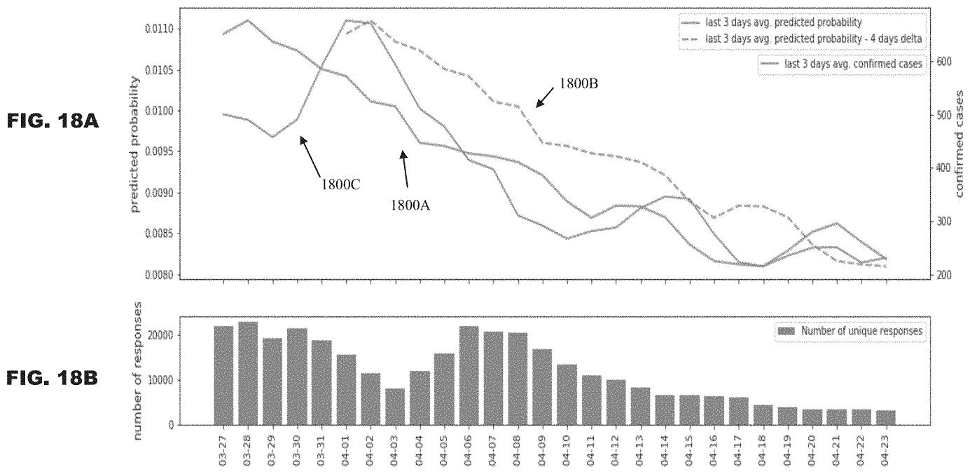

As an additional validation for the risk scores obtained, Inventors compared the model predictions on survey data that was not used in the model construction process (n=87,414), with the actual number of confirmed COVID-19 patients in Israel over time. Notably, Inventors found that the average predicted probability of individuals to test positive for COVID-19 according to the model, is highly correlated with the number of new confirmed COVID-19 cases 4 days later (pearson r=0.90, p<10⁻⁸) (see FIG. 18A-B).

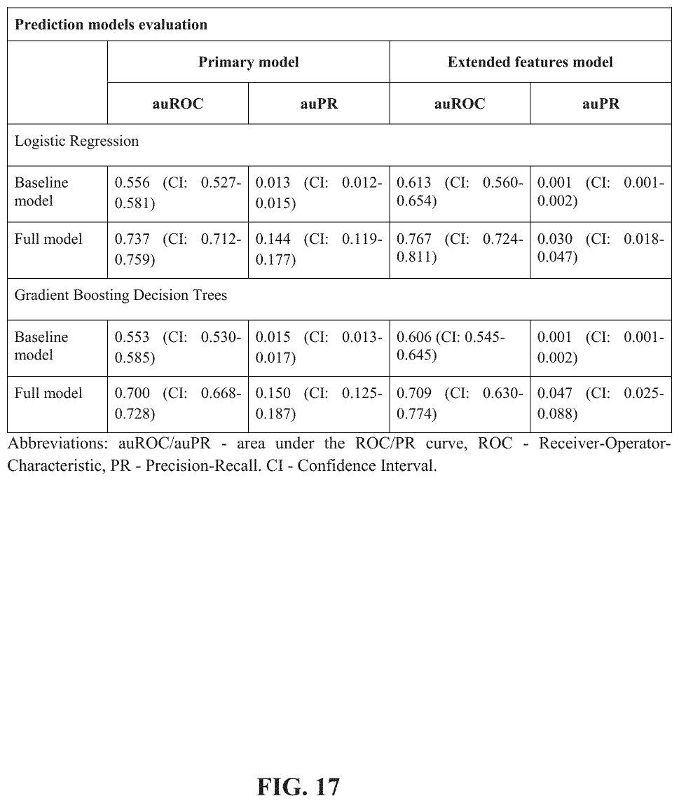

To better understand which features contribute to the probability of being diagnosed with COVID-19, and to examine feature interactions, Inventors analyzed the primary model constructed using the Gradient Boosting Decision Trees algorithm (see Methods section). This model showed similar performance to the Logistic Regression model (see FIG. 16A-F and FIG. 17), with a positive predictive value (PPV) of 51.8%, at 10% sensitivity, and its predictions on online survey data not used in the construction process were highly correlated with the predictions of the primary Logistic Regression model (pearson r=0.91, p<10⁻⁸).

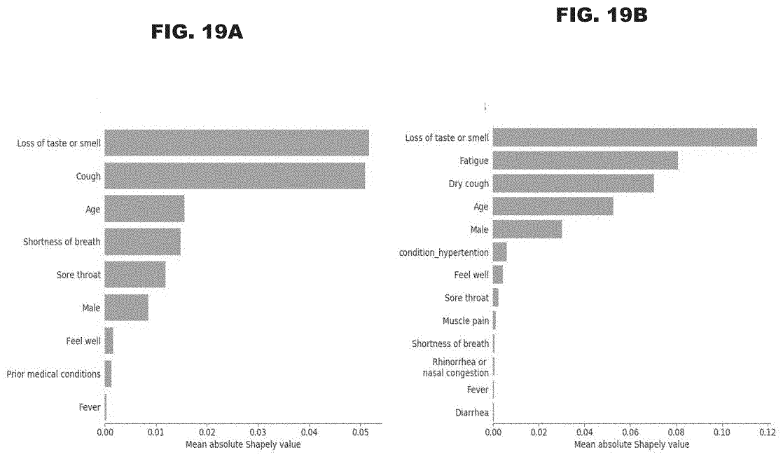

Analysis of feature contribution was performed using Shapley values (see Methods section). Loss of taste or smell and cough had the largest overall contribution to the model (see FIG. 19A), when analyzing the mean absolute SHAP value of features on the entire data. Since the primary model contained a limited number of features, Inventors compared its feature contributions to those obtained from the extended features model, also constructed using a Gradient Boosting Decision Trees algorithm. Notably, loss of taste or smell was the most contributing feature in both the primary model and the extended features model, which contained 14 additional features (see FIG. 19A-B), a fact that is also supported by an odds ratio analysis (see FIG. 24). Sore throat and fever, which were part of the primary model, also ranked highly among the contributing features of the extended features mode (see FIG. 19B). Although the extended features model included 23 features—14 additional features over the primary model, all symptoms included in the primary model were among the 12 features found to be most contributing (see FIG. 19B).

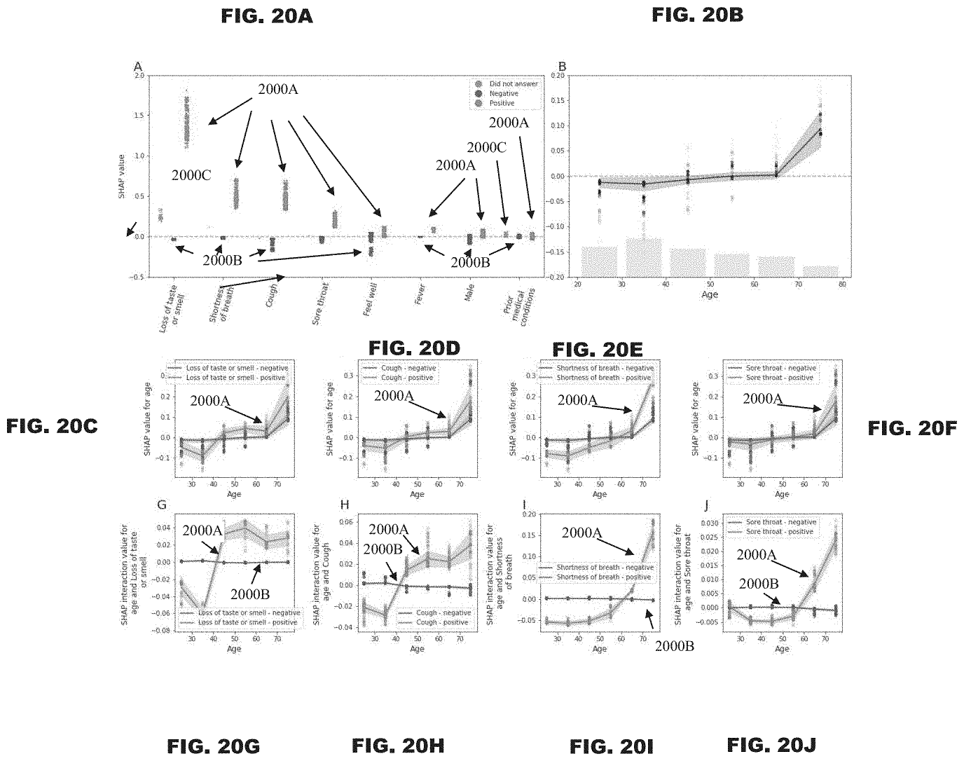

As age was reported as a dominant factor in COVID-19 infection and its clinical manifestation, for example, as described with reference to Harapan H, Itoh N, Yufika A, et al. Coronavirus disease 2019 (COVID-19): A literature review. J Infect Public Health. 2020; 13(5):667-673. doi:10.1016/j.jiph.2020.03.019, Inventors examined the interaction of age with every symptom using SHAP interaction values (see Methods section). For the highest age group (>70 years old), age itself contributes most to the probability of being diagnosed with COVID-19 (see FIG. 20B). Presence of cough and loss of taste or smell exhibits a sharp transition-type (sigmoid-like) interaction with age, such that above the age of 50 years old, presence of each of these symptoms sharply increases the model's predicted probability of COVID-19 infection (see FIG. 20G-H). In contrast, shortness of breath and sore throat show a more gradual (parabolic-like) interaction with age with presence of these symptoms increasing the model's prediction more gradually as the age of the subject being predicted increases (see FIG. 20I-J). Negative answers in all these features show no interaction with age. Other examined features, such as fever and general feeling, do not show such interactions with age.

### DISCUSSION

In this study Inventors constructed an ML model that predicts the probability of individuals to test positive for COVID-19. The ML model is based on 9 simple questions that every person can easily answer in less than a minute from the comfort of her home. The model can assist the worldwide fight against COVID-19 by better prioritizing the limited tests available without additional costs, thereby increasing the rate at which positive individuals can be identified and isolated.

In Israel, as well as in many other countries, due to limited testing resources, suspected patients are only tested if they were exposed to a COVID-19 confirmed patient as well as exhibited acute respiratory symptoms, for example, as described with reference to Home Page, Ministry of Health. www(dot)health(dot)gov(dot)il/English/Pages/HomePage(dot)aspx(dot) Accessed Apr. 29, 2020. By taking an unbiased approach to predicting COVID-19 diagnosis from symptoms data, the analysis described herein highlights the importance of additional features, such as sore throat. Of note, anosmia and ageusia that were less described in patients in the early stages of the COVID-19 pandemic, for example, as described with reference to Zhao X, Zhang B, Li P, et al. Incidence, clinical characteristics and prognostic factor of patients with COVID-19: a systematic review and meta-analysis. medRxiv. January 2020, Gudbjartsson D F, Helgason A, Jonsson H, et al. Spread of SARS-CoV-2 in the Icelandic Population. N Engl J Med. April 2020. doi:10.1056/NEJMoa2006100, Menni C, Valdes A, Freydin M B, et al. Loss of smell and taste in combination with other symptoms is a strong predictor of COVID-19 infection. medRxiv. April 2020. doi:10.1101/2020.04.05.20048421, and Yan C H, Faraji F, Prajapati D P, Boone C E, DeConde A S. Association of chemosensory dysfunction and Covid-19 in patients presenting with influenza-like symptoms. Int Forum Allergy Rhinol. April 2020. doi:10.1002/alr.22579. were the most impactful features in both models for COVID-19 diagnosis. This is inline with current literature demonstrating the importance of these symptoms in early detection and identification of the disease (Menni et al. 2020; Yan et al. 2020), (Lechien et al. 2020). The ML model also successfully recapitulated patterns of the disease that are described in the literature, such as its complex relationship with age, for example, as described with reference to Harapan H, Itoh N, Yufika A, et al. Coronavirus disease 2019 (COVID-19): A literature review. J Infect Public Health. 2020; 13(5): 667-673. doi:10.1016/j.jiph.2020.03.019. In addition, the ML model also unraveled several patterns that are not described in the literature, such as the different patterns of interactions that particular symptoms have with age, suggesting a variation of the clinical manifestation in different age groups. Although the analysis is purely predictive and not necessarily causal, these new patterns may be used to devise better testing policies, and pave the way for future studies that can uncover new aspects of the disease that were not studied to date.

Analysis of the extended features model that included 23 features compared to 9 in the primary model, validated the choice of questions in the shortened version of the survey and suggested that fatigue should also be considered. In addition, the extended features model suggested that while dry cough has an essential role in predicting COVID-19 diagnosis, moist cough does not and may thus help distinguish between cases of COVID-19 and other infections. Some of the most contributing symptoms to the prediction of a COVID-19 diagnosis are currently not included in the testing policy in Israel, for example, as described with reference to Home Page, Ministry of Health. www(dot)health(dot)gov(dot)il/English/Pages/HomePage(dot)aspx(dot) Accessed Apr. 29, 2020, such as loss of smell or taste. The analysis suggests that adding these symptoms to the testing policy should help discriminate which individuals should be tested, and optimize testing priorities.

While informative, the feature contribution analysis may be improved in future Experiments. First, Inventors did not include children in the datasets and thus, symptoms such as nausea or vomiting and diarrhea that were mostly described in children, for example, as described with reference to Dong Y, Mo X, Hu Y, et al. Epidemiological characteristics of 2143 pediatric patients with 2019 coronavirus disease in China. Pediatrics. March 2020. doi:10.1542/peds.2020-0702, and de Souza T H, Nadal J A, Nogueira R J N, Pereira R M, Brandao M B. Clinical Manifestations of Children with COVID-19: a Systematic Review. medRxiv. April 2020. doi:10.1101/2020.04.01.20049833, may have a more significant part in models designed for younger age groups. Second, although Inventors included a large list of prior medical conditions that may have a role in COVID-19 susceptibility, some of these conditions are not highly prevalent in the dataset and their contribution may thus be underestimated in the ML model used for the second set of experiments. Finally, body temperature was the only non-mandatory question in the survey, and may thus have higher predictive power than portrayed within the ML model.

Several studies attempted to simulate and predict different aspects of COVID-19, such as hospital admissions, diagnosis, prognosis and mortality risk, using mostly age, body temperature, medical tests and symptoms, for example, as described with reference to Wynants L, Van Calster B, Bonten M M J, et al. Prediction models for diagnosis and prognosis of covid-19 infection: systematic review and critical appraisal. BMJ. 2020; 369:m1328. doi:10.1136/bmj.m1328. Most diagnostic models published to date were based on datasets from China and included complex features that had to be extracted through blood tests and imaging scans, for example, as described with reference to Wynants L, Van Calster B, Bonten M M J, et al. Prediction models for diagnosis and prognosis of covid-19 infection: systematic review and critical appraisal. BMJ. 2020; 369:m1328. doi:10.1136/bmj.m1328. As described herein, Inventors devised a prediction model which was based solely on self-reported information (or reported by others), and as such it could be easily deployed and used in a short time. Although survey collected data may suffer from response bias, the IVR collected responses were collected by active contact with all city residents in chosen cities and resulted in a high response rate of up to 16% of the residents in some cities.

The second set of Experiments may have several additional limitations, which do not necessarily detract from the results described herein (e.g., high accuracy of the ML model), but which may be improved during subsequent Experiments. First, the data is biased by Israel's MOH ever changing testing policy, such that at some point all of the COVID-19 positively diagnosed participants in the study had to be eligible for a test under that policy. An ideal dataset for purposes of devising a classifier should include a large random sampling of the population, but such data coupled with symptom surveys is currently unavailable at large-scale. Accordingly, all the diagnosed responders in the study are not in the first stage of showing symptoms, but are in some time-lag after diagnosis. In addition, the study is based on self reports of willing participants and is therefore bound to suffer from some selection bias. The bias is significantly reduced in the data collected via the IVR platform, since all residents in the IVR-surveyed cities were actively contacted only once, on the same day and in the same manner. In the online version of the survey Inventors made attempts to reduce this bias by promoting it in several media outlets and by engaging leaders of underrepresented communities.

In conclusion, Inventors' constructed ML model predicts COVID-19 PCR test results with high discrimination (positive predictive value (PPV) of 51.8% at a 10% sensitivity) and calibration. It also suggests that several symptoms that are currently not included in the Israeli testing policy exhibit intriguing interactions with age and should probably be integrated into revised testing policies. Overall, at least some of the systems, methods, apparatus, and/or code instructions described herein may be utilized worldwide to direct the limited resources towards individuals who are more likely to test positive for COVID-19, leading to faster isolation of infected patients and therefore to reduced rates of spreading of the virus.

Reference is now made to FIGS. 14-25, which present data used in the second set of experiments and results of the second set of experiments, in accordance with some embodiments of the present invention.

FIG. 14 is a study population flow chart for the second set of experiments, in accordance with some embodiments of the present invention. Numbers represent recorded responses. Boxes 1402 and 1404 show responses which were used in extended features model (top) and primary model (bottom) constructions.

FIG. 15 is a table of baseline characteristics of the primary model population for the second set of experiments, in accordance with some embodiments of the present invention.

FIGS. 16A-F are graphs presenting performance of the primary model for the second set of experiments, in accordance with some embodiments of the present invention. FIG. 16A-C: Logistic Regression, FIG. 16D-F: Gradient Boosting Decision Trees. FIG. 16A, D: ROC curve of Inventor's model (blue 1600A) consisting of 9 simple questions and of the baseline model consisting of only age and gender (red 1600B). Different decision probability thresholds are marked on the curve. FIG. 16B, E: Precision-Recall curve of Inventor's model (blue 1600A) and the baseline model (red 1600B). Different decision probability thresholds are marked on the curve. FIG. 16C, F: Calibration curve. Dots 1600C represent deciles of predicted probabilities. Dotted diagonal line 1600D represents an ideal calibration. Bottom: Log-scaled histogram of predicted probabilities of COVID-19 undiagnosed (bar graph on left side of each pair of bars) and diagnosed (bar graph of right side of each pair of bar).

FIG. 17 is a table presenting evaluations for the primary model and the extended features model for the second set of experiments, in accordance with some embodiments of the present invention.

FIGS. 18A-B are graphs depicting a comparison of the primary model predictions to new COVID-19 cases in Israel over time, for the second set of experiments, in accordance with some embodiments of the present invention. FIG. 18A: Primary model predictions, averaged across all individuals in on a 3-day running average (solid blue 1800A), and shifted 4 days forward (dotted blue 1800B), compared to the number of newly confirmed COVID-19 cases in Israel by the ministry of health (MOH), based on a 3-day running average 1800 C. FIG. 18B: Number of survey responses per day.

FIG. 19A-B are graphs depicting a feature contribution analysis, for the second set of experiments, in accordance with some embodiments of the present invention. Mean absolute Shapley value (in units of log-odds) of the Primary model, including all features used in the model (19A), and the Extended features model, for the 13 highest contributing features (19B).

FIG. 20A-J are graphs depicting a feature interpretation analysis, for the second set of experiments, in accordance with some embodiments of the present invention. FIG. 20A: SHAP values (in units of log-odds) for all features, with positive answers colored red 2000A, negative colored in blue 2000B and missing answers in grey 2000C. FIG. 20B: SHAP values for age with number of responses histogram at the bottom. FIG. 20C-F: SHAP value for age, stratified by positive (red 2000A) and negative (blue 2000B) responses of loss of taste or smell (C), cough (D), shortness of breath (E) and sore throat (F). FIG. 20G-J: SHAP interaction values of positive (red 2000A) and negative (blue 2000B) responses with age, of loss of taste or smell (G), cough (H), shortness of breath (I) and sore throat (J).

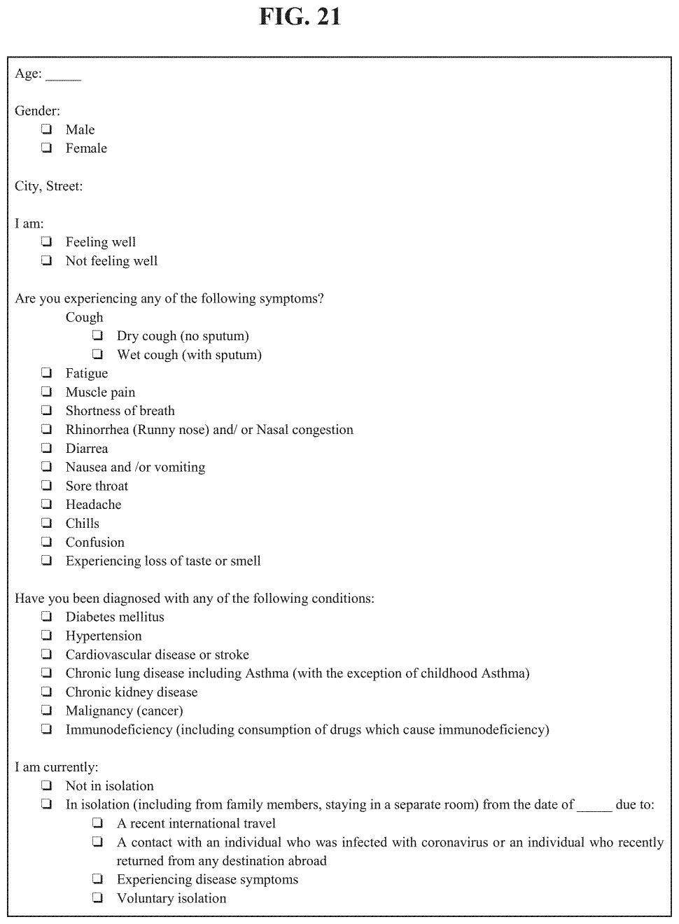

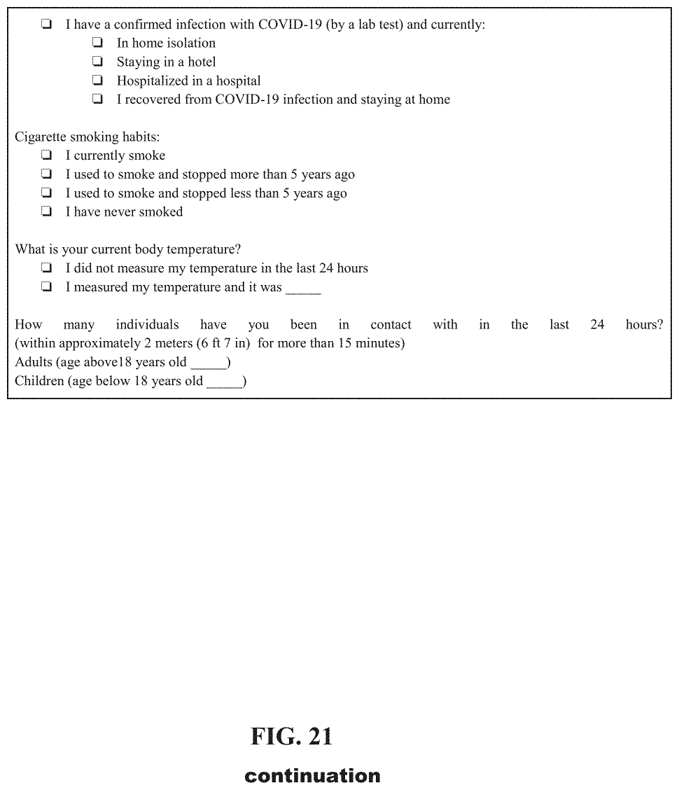

FIG. 21 is the online version questions for the COVID-19 survey, for the second set of experiments, in accordance with some embodiments of the present invention.

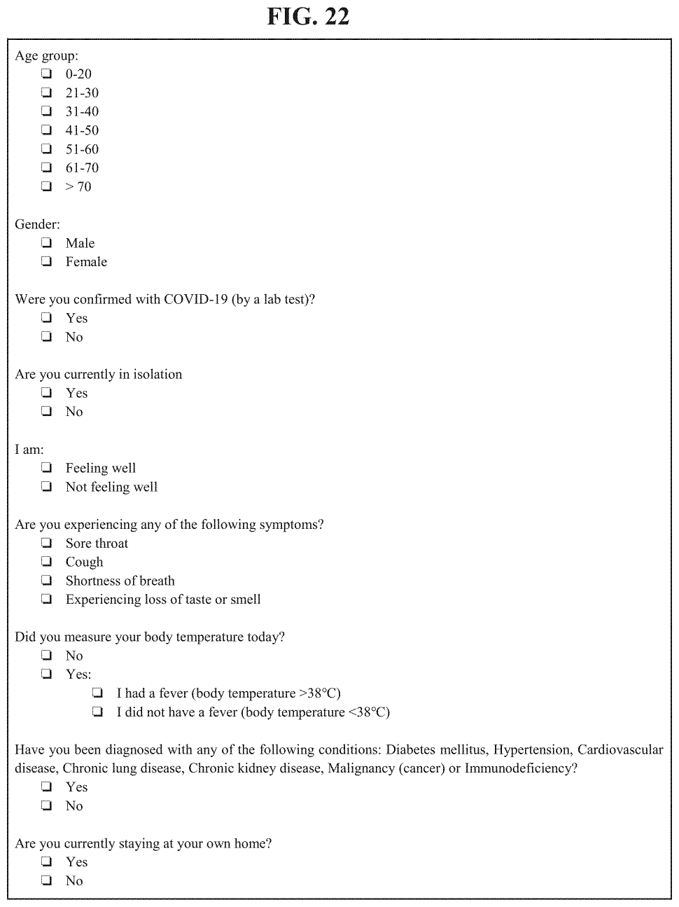

FIG. 22 is the IVR version questions, for the second set of experiments, in accordance with some embodiments of the present invention.

FIG. 23 is a table presenting COVID-19 diagnosis prevalence and response rate in the IVR cities, for the second set of experiments, in accordance with some embodiments of the present invention.

FIG. 24 is a table presenting baseline characteristics of the extended features model population, for the second set of experiments, in accordance with some embodiments of the present invention.

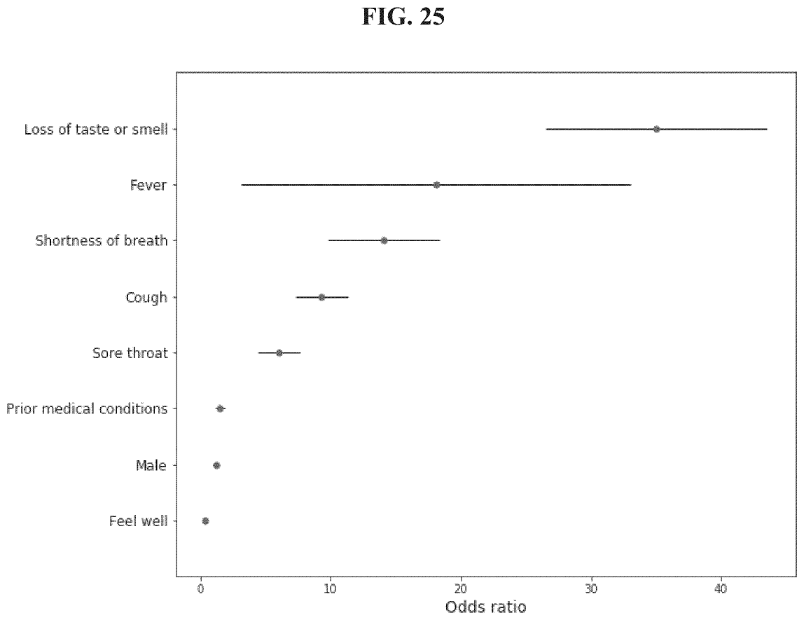

FIG. 25 is a chart presenting an odd-ratio (unadjusted) analysis of the primary model population, for the second set of experiments, in accordance with some embodiments of the present invention.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

It is expected that during the life of a patent maturing from this application many relevant ML models will be developed and the scope of the term ML model is intended to include all such new technologies a priori.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

What is claimed is:

1. A computer implemented method for diagnosing pathogenesis of viral infections for epidemic prevention, comprising:
receiving a plurality of responses to a questionnaire provided to a plurality of undiagnosed persons, each of the plurality of responses comprises a plurality of answers and associated with a geographical location within one of a plurality of geographic zones;
in each of a plurality of iterations:
inputting a first subset of the plurality of answers and the geographical location of one of the plurality of undiagnosed persons into a geographic-level machine learning (ML) model component trained on a first training dataset including, for each of a plurality of subjects: the first subset of the plurality of answers, an indication of a certain geographic zone of the plurality of geographic zones, and a label indicative of whether the respective subject is diagnosed or undiagnosed with the viral disease;
inputting a second subset of the plurality of answers to a human-level ML model component trained on a second training dataset including, for each of a plurality of subjects, the second subset of the plurality of answers, and a label indicative of whether the respective subject is diagnosed or undiagnosed with the viral disease;
combining the outcome from the ML model components to calculate a combined likelihood of the respective undiagnosed person to be diagnosed with the viral disease; and
treating the respective undiagnosed person for the viral disease according to the combined likelihood using a treatment effective for the viral disease.

2. The method of claim 1, wherein the viral disease comprises COVID-19.

3. The method of claim 1, wherein for at least some of the plurality of undiagnosed persons all of the answers are indicative of lack of any symptoms correlated with the viral disease indicating that each one of the at least some of the plurality of undiagnosed persons are asymptomatic.

4. The method of claim 1, wherein the subject is treated for the viral disease with an effective treatment when the combined likelihood is above a threshold.

5. The method of claim 4, wherein the viral disease comprises COVID-19 and the effective treatment is selected from the group consisting of: mechanical ventilation, supplemental oxygen, respiratory support, antipyretics, anti-virals, Remdesivir, Oseltamivir, steroids, plasma including antibodies to COVID-19 of subjects that recovered from COVID-19, chloroquine, hydroxychloroquine, and a vaccine against COVID-19.

6. The method of claim 1, further comprising computing a symptom score by aggregating answers indicative of symptoms, and wherein inputting the first and second subset comprises at least one of: inputting the symptom score, and inputting the symptom score in addition to inputting the first subset and the second subset of answers.

7. The method of claim 6, wherein the symptom score is computed as a number of positive answers indicative of presence of symptoms, divided by a total number of questions indicative of possible symptoms.

8. The method of claim 1, wherein the questionnaire includes questions denoting presence of symptoms correlated with population-level likelihood of being infected with the viral disease.

9. The method of claim 8, wherein presence of the symptoms represented by the plurality of answers to the questions are selected from the group consisting of: no symptoms and feeling good, body temperature, body temperature greater than a threshold, nausea and vomiting, myalgia, rhinorrhea or nasal congestion, fatigue, shortness of breath, cough, sore throat and loss of taste or smell, dry cough, moist cough, chills, confusion, a certain prior medical condition, and diarrhea.

10. The method of claim 1, wherein the questionnaire includes questions denoting presence of symptoms negatively correlated with population-level likelihood of being infected with the viral disease and positively correlated with population-level likelihood of having another medical condition unrelated to the viral disease.

11. The method of claim 1, wherein a certain answer to a certain question comprises at least one of:
(i) an age of the undiagnosed subject, and further comprising including the age in at least one of the first subset and second subset, and
(ii) smoking history and/or presence of chronic medical conditions of the undiagnosed subject, and including the smoking history and/or presence of chronic medical conditions into at least one of the first and second subsets.

12. The method of claim 1, further comprising receiving for at least some of the plurality of undiagnosed persons, the plurality of answers for a plurality of questionnaires obtained at sequential time intervals, and including the plurality of responses to the plurality of questions for the plurality of questionnaires obtained at sequential time intervals in at least one of the first and second subsets.

13. The method of claim 12, wherein a combination of non-symptom related questions of the questionnaire denote a unique identifier of each respective undiagnosed person of the at least some of the plurality of undiagnosed persons, and further comprising arranging the responses into the sequential time interval for each respective undiagnosed person according to the unique identifier based on a unique combination of answers to the combination of non-symptom related questions.

14. The method of claim 1, further comprising receiving an indication of dynamic flow of subjects between the certain geographical zone and at least one other geographical zone, and inputting the indication of dynamic flow into the geographic-level ML model component.

15. The method of claim 14, wherein the indication of dynamic flow is selected from the group consisting of: traffic patterns, public transportation routes, and walking patterns of subjects.

16. The method of claim 1, further comprising receiving an indication of dynamic flow of the undiagnosed subject between the certain geographical zone and at least one other geographical zone, and inputting the indication of dynamic flow into at least one of: the geographic-level ML model component and the human-level ML model component.

17. The method of claim 1, further comprising receiving at least one supplementary static and/or dynamic data, and inputting the at least one supplementary static and/or dynamic data into at least one of: the geographic-level ML model component and the human-level ML model component, wherein the at least one supplementary data is selected from the group consisting of: meteorological data within geographical zones, prescriptions of medications correlated with the viral disease within geographical zones, population density of geographical zones, locations of educational institutions within geographical zones, locations of religious houses of worship within geographical zones, locations of shopping malls within geographical zones, hospitalization of subjects living within geographical zones, subjects assigned to quarantine within geographical zones.

18. The method of claim 1, wherein the outcome from the ML model components is inputted into a combination ML model component that is trained on a third training dataset including, for each of a plurality of subjects, output of the human-level ML component and output of the geographic-level ML component, and a label indicative of whether the respective subject is diagnosed or undiagnosed with the viral disease.

19. The method of claim 1, wherein the first subset of the plurality of answers and the geographical location are inputted into a first processing path comprising the geographic-level ML model component, the second subset of the plurality of answers are inputted into a second processing path comprising the human-level ML model component, and the combination of the outcomes from the ML model components is inputted into a third combined processing path.

20. The method of claim 1, wherein the human level-ML component outputs a human-level likelihood of a certain undiagnosed person likely to be diagnosed with the viral disease.

21. The method of claim 1, wherein the geographic-level ML component outputs at least one of: a geographic-level prediction for number of undiagnosed persons within each respective geographic zone likely to be diagnosed with the viral disease, and a geographic-level prediction for a percent of undiagnosed persons within each respective geographic zone likely to be diagnosed with the viral disease.

22. The method of claim 1, further comprising: obtaining, for each of the plurality of undiagnosed persons for each of the plurality of geographic zones, a respective combined likelihood, aggregating for each geographic zone the combined likelihoods of undiagnosed persons located within the respective geographic zone, and creating and presenting a coded map indicating, computing a number and/or a percentage of undiagnosed persons likely being diagnosed with the viral disease for each of the plurality of geographic zones.

23. The method of claim 22, further comprising creating and presenting a coded map indicating the number and/or the percentage of undiagnosed persons likely being diagnosed with the viral disease for each of the plurality of geographic zones.

24. The method of claim 22, wherein the respective combined likelihood is computed based on the plurality of responses to the questionnaire provided to the plurality of undiagnosed persons on a certain day.

25. The method of claim 22, further comprising aggregating the combined likelihoods of undiagnosed persons for the plurality of geographic zones for computing a number and/or a percentage of undiagnosed persons likely being diagnosed with the viral disease for a large area consisting of the plurality of geographic zones.

26. The method of claim 1, wherein a first subset of the plurality of answers and the geographical location of the plurality of undiagnosed persons are inputted into the geographic-level ML model, and a unified geographic-level outcome is obtained from the geographic-level ML model component; and
wherein combining comprises combining, for each of the plurality of iterations, the geographic-level outcome from the geographic-level ML model component and each respective outcome from the human-level ML model component, to calculate a respective combined likelihood of the respective undiagnosed person to be diagnosed with the viral disease.

27. A computer implemented method for training an ML model for classifying people in multiple geographic areas, comprising:
obtaining, for each respective subject of a plurality of subjects, a plurality of responses to a questionnaire provided to a plurality of undiagnosed persons, each of the plurality of responses comprises a plurality of answers, an indication of a certain geographic zone of a plurality of geographic zones, and a label indicative of whether the respective subject is diagnosed or undiagnosed with the viral disease;
creating a geographic-level training dataset including, for each of the plurality of subjects: a first subset of the plurality of answers, the indication of the certain geographic zone of the plurality of geographic zones, and the label indicative of whether the respective subject is diagnosed or undiagnosed with the viral disease;
training the geographic-level ML model component using the geographic-level training dataset;
creating a human-level training dataset that includes for each of the plurality of subjects, a second subset of the plurality of answers, and the label indicative of whether the respective subject is diagnosed or undiagnosed with the viral disease;
training the human-level ML model component using the human-level training dataset;
computing a combination ML model component that combines the outcome from the geographic-level and the human-level ML model components to calculate a combined likelihood of a target undiagnosed person to be diagnosed with the viral disease; and
providing the ML model that includes the geographic-level ML model component, the human-level ML model component, and the combination ML model component.

28. A method for classifying people in multiple geographic areas, comprising:
receiving a plurality of responses to a questionnaire provided to a plurality of undiagnosed persons, each of the plurality of responses comprises a plurality of answers and associated with a geographical location within one of a plurality of geographic zones;
in each of a plurality of iterations:
analyzing a first subset of the plurality of answers and the geographical location of one of the plurality of undiagnosed persons;
analyzing a second subset of the plurality of answers;
combining the outcomes from the analysis to calculate a combined likelihood of the respective undiagnosed person to be diagnosed with the viral disease; and
treating the respective undiagnosed person for the viral disease according to the combined likelihood using a treatment effective for the viral disease.

29. The method of claim 28, wherein at least one of: a geographic-level prediction for number of undiagnosed persons within each respective geographic zone likely to be diagnosed with the viral disease, and a geographic-level prediction for a percent of undiagnosed persons within each respective geographic zone likely to be diagnosed with the viral disease, is obtained by the analysis of the first subset of the plurality of answers and the geographical location of one of the plurality of diagnosed persons is analyzed.

30. The method of claim 28, wherein a human-level likelihood of the one of the plurality of undiagnosed persons likely to be diagnosed with the viral disease is obtained by the analysis of the second subset of the plurality of answers.
